(19) United States
(12) Patent Application Publication
(10) Pub. No.: US 2023/0165904 A1
(43) Pub. Date: Jun. 1, 2023

(54) METHOD FOR TREATING HYPERINFLAMMATION USING MESENCHYMAL LINEAGE PRECURSOR OR STEM CELLS

(71) Applicant: Mesoblast International Sárl, Meyrin (CH)

(72) Inventor: Silviu ITESCU, Melbourne (AU)

(21) Appl. No.: 17/995,385

(22) PCT Filed: Apr. 1, 2021
(86) PCT No.: PCT/EP2021/058683

§ 371 Date: Oct. 3, 2022

(30) Foreign Application Priority Data
Apr. 3, 2020 (AU) 2020901052
Apr. 8, 2020 (AU) 2020901124
Jul. 6, 2020 (AU) 2020902312
Jul. 14, 2020 (AU) 2020902425
Aug. 25, 2020 (AU) 2020903041
Oct. 12, 2020 (AU) 2020903694
Nov. 23, 2020 (AU) 2020904312

(51) Int. Cl. A61K 35/28; A61P 29/00

(52) U.S. Cl. CPC A61K 35/28; A61P 29/00

(57) ABSTRACT

The present disclosure relates to method of treating or preventing hyperinflammation in a subject in need thereof, the method comprising administering to the subject a composition comprising mesenchymal lineage precursor or stem cells (MLPSCs).

### FIELD OF THE DISCLOSURE

The present disclosure relates to methods for treating or preventing hyperinflammation in a subject in need thereof.

### BACKGROUND

Respiratory ailments, associated with a variety of conditions such as viral infection are problematic in the general population. In many cases they are accompanied by hyperinflammation, which aggravates the condition of the lungs.

Accordingly, there remains an unmet therapeutic need in patients with hyperinflammation, particularly when secondary to viral infection with new treatment options being required.

### SUMMARY OF THE DISCLOSURE

The present inventors have surprisingly identified that treatment of hyperinflammation can be achieved by administering mesenchymal lineage precursor or stem cells (MLPSCs).

Accordingly, in a first example, the present disclosure relates to a method of treating or preventing hyperinflammation in a human subject in need thereof, the method comprising administering to the subject a composition comprising mesenchymal lineage precursor or stem cells (MLPSCs).

In an example, the hyperinflammation is caused by a viral infection. The viral infection may be caused, for example, by a rhinovirus, influenza virus, respiratory syncytial virus (RSV) or a coronavirus.

In one example, the hyperinflammation is caused by a coronavirus infection. The coronavirus may be, for example, Severe Acute Respiratory Syndrome coronavirus (SARS-CoV), Middle East Respiratory Syndrome coronavirus (MERS-CoV), COVID-19, 229E, NL63, OC43, or KHU1. In one example, the coronavirus is SARS-CoV, MERS-CoV or COVID-19 (SARS-CoV-2).

In another example, the subject also has Acute Respiratory Distress Syndrome (ARDS). In another example, the subject has multi-system inflammatory syndrome. In this example, the subject may be less than 21 years old. In an example, the subject with MIS is a child. In another example, the subject has viral myocarditis.

Accordingly, in an example, the present disclosure relates to a method of treating ARDS in a human subject in need thereof, the method comprising administering to the subject a composition comprising mesenchymal lineage precursor or stem cells (MLPSCs).

In an example, the MLPSCs have been cryopreserved and thawed. In an example, the MLPSCs are culture expanded from an intermediate cryopreserved MLPSCs population. In another example, the MLPSCs are culture expanded for at least about 5 passages. In an example, the MLPSCs express at least 13 pg TNFR1 per million MLPSCs. In an example, the MLPSCs express about 13 pg to about 44 pg TNFR1 per million MLPSCs. In an example, culture expanded MLPSCs are culture expanded for at least 20 population doublings. In another example, culture expanded MLPSCs are culture expanded for at least 30 population doublings. In an example, the MLPSCs are mesenchymal stem cells (MSCs). In another example, the MLPSCs are allogeneic. For example, the MLPSCs may be allogeneic MSCs.

In another example, the MLPSCs are modified to carry or express an anti-viral drug or a thrombolytic agent. In an example, the anti-viral drug is Remdesivir. In an example, the thrombolytic agent is selected from the group consisting of Eminase (anistreplase) Retavase (reteplase) Streptase (streptokinase, kabikinase).

In another example, the MLPSCs are genetically modified to express an anti-viral peptide or a nucleic acid encoding the same.

In an example, the composition is administered intravenously.

In an example, the methods of the disclosure encompass administering between 1×10⁷ and 2×10⁸ cells. For example, multiple doses of between 1×10⁷ and 2×10⁸ cells may be administered on days 0, 30, 60 and 90. In an example, the methods of the disclosure encompass administering about 1×10⁸ cells per dose. In an example, the subject is administered two doses.

In an example, the subjects circulating CRP levels decrease after treatment. In an example, the subjects white blood cell levels decrease after treatment. In an example, the subjects D-dimer level is reduced after treatment. In an example, the subjects D-dimer level is reduced below 5 ug/ml. In an example, the subjects BNP level is reduced after treatment. In an example, the subjects FiO2 levels decrease after treatment. In an example, the subjects P/F ratio increases after treatment. In an example, the subjects LVEF % is increased after treatment.

In another example, the composition further comprises Plasma-Lyte A, dimethyl sulfoxide (DMSO), human serum albumin (HSA). In an example, the composition further comprises Plasma-Lyte A (70%), DMSO (10%), HSA (25%) solution, the HSA solution comprising 5% HSA and 15% buffer. In an example, the composition comprises greater than 6.68×10⁶ viable cells/mL.

In another example, the present disclosure relates to a method of treating or preventing multi-system inflammatory syndrome (MIS) in a human subject in need thereof, the method comprising administering to the subject a composition comprising mesenchymal lineage precursor or stem cells (MLPSCs). In an example, the subject may be a child.

In another example, the present disclosure relates to a method of treating or preventing a disease associated with elevated D-dimer levels in a human subject in need thereof, the method comprising administering to the subject a composition comprising mesenchymal lineage precursor or stem cells (MLPSCs). In an example, the disease is caused by a venous blockage. In an example, the disease is caused by an arterial blockage. In an example, the disease is caused by thrombosis. In an example, the disease is caused by pulmonary embolism. In an example, treatment reduces the subjects D-dimer level below 15 μg/ml. In an example, the subject has a LVEF of less than 55% prior to treatment. In an example, the subject has a LVEF of less than 52% prior to treatment. In another example, the subject has a BNP level greater than 400 pg/ml. In another example, the subject has a BNP level greater than 500 pg/ml.

In another example, the present disclosure relates to a method of treating or preventing a disease associated with elevated D-dimer levels in a human subject in need thereof, the method comprising administering to the subject a composition comprising mesenchymal lineage precursor or stem cells (MLPSCs). In an example, the disease is thrombosis or embolism. In an example, the thrombosis is arterial thrombosis. In an example, the embolism is pulmonary embolism.

In another example, the present disclosure relates to a method of treating or preventing thrombosis in a human subject in need thereof, the method comprising administering to the subject a composition comprising mesenchymal lineage precursor or stem cells (MLPSCs). In an example, the thrombosis is a venous thrombosis. In another example, the thrombosis is an arterial thrombosis. In another example, the present disclosure relates to a method of treating or preventing pulmonary embolism in a human subject in need thereof, the method comprising administering to the subject a composition comprising mesenchymal lineage precursor or stem cells (MLPSCs). In an example, treatment reduces the subjects D-dimer level below 15 μg/ml. In an example, the subject has a LVEF of less than 55% prior to treatment. In an example, the subject has a LVEF of less than 52% prior to treatment. In another example, the subject has a BNP level greater than 400 pg/ml. In another example, the subject has a BNP level greater than 500 pg/ml. In an example, the thrombosis is arterial thrombosis.

In an example, administered MLPSCs are mesenchymal stem cells (MSCs). In an example, the MLPSCs are allogeneic.

### BRIEF DESCRIPTION OF ACCOMPANYING FIGURES

FIG. 1: Preliminary result 1—PaO2/FiO2; White blood cell count.

FIG. 2: Preliminary result 1—CRP; Ferritin.

FIG. 3: Preliminary result 3—PaO2/FiO2; White blood cell count.

FIG. 4: Preliminary result 3—CRP; Ferritin.

FIG. 5: Result summary.

FIG. 6: Nadir PaO2/FiO2 ratios (n=5).

FIG. 7: Median PaO2/FiO2 ratios (n=5).

FIG. 8: Circulating CRP levels (n=5).

FIG. 9: Creatinine levels (n=5).

FIG. 10: Ferritin levels (n=5).

FIG. 11: Swimmer Plot of events according to time from cell therapy infusion. The numbers on the left represent individual patient identifiers as indicated in Table 2.

FIG. 12: Median P:F Ratio and Interquartile Range according to time from cell therapy infusion

FIG. 13: Median C-Reactive protein levels and interquartile range according to time from cell therapy infusion.

FIG. 14: Detailed clinical course; Patient 1.

FIG. 15: Detailed clinical course; Patient 2.

FIG. 16: Comparison of LVEF %, BNP (pg/ml) and D-Dimer (ug/ml) levels.

### DETAILED DESCRIPTION

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

Those skilled in the art will appreciate that the disclosure described herein is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific embodiments described herein, which are intended for the purpose of exemplification only. Functionally-equivalent products, compositions and methods are clearly within the scope of the disclosure, as described herein.

Any example disclosed herein shall be taken to apply mutatis mutandis to any other example unless specifically stated otherwise.

Unless specifically defined otherwise, all technical and scientific terms used herein shall be taken to have the same meaning as commonly understood by one of ordinary skill in the art (e.g., in cell culture, molecular genetics, stem cell differentiation, immunology, immunohistochemistry, protein chemistry, and biochemistry).

Unless otherwise indicated, the surgical techniques utilized in the present disclosure are standard procedures, well known to those skilled in the art.

Methods of obtaining and enriching a population of mesenchymal lineage stem or precursor cells are known in the art. For example, enriched populations of mesenchymal lineage stem or precursor cells can be obtained by the use of flow cytometry and cell sorting procedures based on the use of cell surface markers that are expressed on mesenchymal lineage stem or precursor cells.

All documents cited or referenced herein, and all documents cited or referenced in herein cited documents, together with any manufacturer's instructions, descriptions, product specifications, and product sheets for any products mentioned herein or in any document incorporated by reference herein, are hereby incorporated herein by reference in their entirety.

### Selected Definitions

The term “and/or”, e.g., “X and/or Y” shall be understood to mean either “X and Y” or “X or Y” and shall be taken to provide explicit support for both meanings or for either meaning.

As used herein, the term about, unless stated to the contrary, refers to +/−10%, more preferably +/−5%, of the designated value.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein, the singular form “a”, “an” and “the” include singular and plural references unless the context indicates otherwise.

By “isolated” or “purified” it is meant a cell which has been separated from at least some components of its natural environment. This term includes gross physical separation of the cells from its natural environment (e.g. removal from a donor). The term “isolated” includes alteration of the cell's relationship with the neighboring cells with which it is in direct by, for example, dissociation. The term “isolated” does not refer to a cell which is in a tissue section. When used to refer to the population of cells, the term “isolated” includes populations of cells which result from proliferation of the isolated cells of the disclosure.

The terms “passage”, “passaging” or “sub-culture” are used in the context of the present disclosure to refer to known cell culture techniques that are used to keep cells alive and growing under cultured conditions for extended periods of time so that cell numbers can continually increase. The degree of sub-culturing a cell line has undergone is often expressed as “passage number,” which is generally used to refer to the number of times cells have been sub-cultured. In an example, one passage comprises removing non-adherent cells and leaving adherent mesenchymal lineage precursor or stem cells. Such mesenchymal lineage precursor or stem cells can then be dissociated from the substrate or flask (e.g., by using a protease such as trypsin or collagenase), media can be added, optional washing (e.g., by centrifugation) may be performed, and then the mesenchymal lineage precursor or stem cells can be re-plated or reseeded to one or more culture vessels containing a greater surface area in total. The mesenchymal lineage precursor or stem cells can then continue to expand in culture. In another example, methods of removing non-adherent cells include steps of non-enzymatic treatment (e.g., with EDTA). In an example, mesenchymal lineage precursor or stem cells are passaged at or near confluence (e.g., about 75% to about 95% confluence). In an example, the mesenchymal lineage precursor or stem cells are seeded at a concentration of about 10%, about 15%, or about 20% cells/ml of culture medium.

The term “medium” or “media” as used in the context of the present disclosure, includes the components of the environment surrounding cells in culture. It is envisaged that the media contributes to and/or provides the conditions suitable to allow cells to grow. Media may be solid, liquid, gaseous or a mixture of phases and materials. Media can include liquid growth media as well as liquid media that do not sustain cell growth. Exemplary gaseous media include the gaseous phase that cells growing on a petri dish or other solid or semisolid support are exposed to.

As used herein, the terms “treating”, “treat” or “treatment” include administering a population of mesenchymal lineage stem or precursor cells and/or progeny thereof and/or soluble factors derived therefrom to thereby reduce or eliminate at least one symptom of hyperinflammation. In an example, treatment includes administering a population of culture expanded mesenchymal lineage stem or precursor cells. In an example, treatment response is determined relative to baseline.

In an example, treatment is determined based on a subjects CRP level. In other examples, treatment is determined based on one or more of white blood cell count, P/F ratio or ferritin levels. In an example, treatment improves P/F by 50 compared to baseline. In another example, treatment increases a subjects P/F above 200. In another example, treatment increases a subjects P/F above 250.

“C-reactive protein” or “CRP” is an inflammatory mediator whose levels are raised under conditions of acute inflammatory recurrence and rapidly normalize once the inflammation subsides.

In an example, treatment is determined based on a reduction in CRP, Procalcitonin (PCT) and ferritin levels.

In an example, treatment reduces CRP by at least 100 mg/dl compared to baseline. In another example, treatment reduces CRP by at least 150 mg/dl compared to baseline. In an example, a decrease in circulating CRP levels is a reduction in CRP to 80 mg/dl or lower. In an example, a decrease in circulating CRP levels is a reduction in CRP to 60 mg/dl or lower. In another example, a decrease in circulating CRP levels is a reduction in CRP to 50 mg/dl or lower. In another example, a decrease in circulating CRP levels is a reduction in CRP to 40 mg/dl or lower. In another example, a decrease in circulating CRP levels is a reduction in CRP to 20 mg/dl or lower. In another example, a decrease in circulating CRP levels is a reduction in CRP to 10 mg/dl or lower. In another example, a decrease in circulating CRP levels is a reduction in CRP to 5 mg/dl or lower. In another example, a decrease in circulating CRP levels is a reduction in CRP to 3 mg/dl or lower.

In another example, treatment reduces CRP to between 0.5 mg/dl and 60 mg/dl. In another example, treatment reduces CRP to between 0.5 mg/dl and 30 mg/dl. In another example, treatment reduces CRP to between 0.5 mg/dl and 10 mg/dl.

In an example, treatment decreases ferritin levels below 2000 mg/dl. In another example, treatment decreases ferritin levels by at least 500 mg/dl compared to baseline. In another example, treatment decreases ferritin levels by at least 750 mg/ml compared to baseline. In another example, treatment decreases ferritin levels below 700 mg/dl.

In another example, treatment decreases PCT levels below 0.12 ng/ml.

In another example, treatment decreases PCT levels below 0.1 ng/ml. In another example, treatment decreases PCT levels below 0.08 ng/ml.

In an example, in subjects with ARDS, treatment improves the subjects ARDS from moderate to severe. In this example, treatment improves a subjects P/F by at least 50 compared to baseline. In an example, treatment improves a subject P/F above 200.

In an example, treatment decreases IL-6 levels. In another example, treatment is based on a change in triglyceride levels.

In an example, treatment is determined based on spirometry measurements. In an example, spirometry measurements are determined based on American Association for Respiratory Care (AARC) Spirometry Clinical Practice Guideline (American Association for Respiratory Care: AARC clinical practice guideline: Spirometry, 1996 Update., Respir Care., 41:629-638).

In an example, treatment improves a subjects FEV1. In an example, treatment improves a subjects FVC. In another example, treatment improves a subjects FEV1/FVC. In another example, treatment improves a subjects 6 minute walk test.

In an example, 6 minute walk test results are determined based on ATS statement: guidelines for the six-minute walk test (2002) Am J Respir Crit Care Med., 166:111-7.

“D-dimer” is one of the circulating terminal breakdown products of Fibrin formed following the action of Plasmin on a clot. D-dimer levels are a marker of major thrombosis and clotting. In an example, treatment according to the disclosure reduces D-dimer levels in a subject and therefore reduces the risk of thrombosis and clotting. In an example, treatment reduces a subjects D-dimer level. In an example, treatment reduces a subjects D-dimer level below 25 μg/ml. In an example, treatment reduces a subjects D-dimer level below 20 μg/ml. In an example, treatment reduces a subjects D-dimer level below 15 μg/ml. In an example, treatment reduces a subjects D-dimer level below 10 μg/ml. In an example, treatment reduces a subjects D-dimer level below 5 μg/ml. In an example, treatment reduces a subjects D-dimer level below 3 μg/ml. In an example, treatment reduces a subjects D-dimer level to between 1 μg/ml and 15 μg/ml. In an example, treatment reduces a subjects D-dimer level to between 1 μg/ml and 10 μg/ml.

In an example, treatment according to the methods of the present disclosure reduce a subjects risk of thrombosis. In an example, the subjects risk is reduced relative to a subject that does not receive treatment. In an example, treatment reduces the risk of the thrombosis is arterial thrombosis. Accordingly, in an example, treatment reduces the risk of heart attack or stroke.

“B-type natriuretic peptide” or “BNP” is a hormone produced by the heart which is released in response to changes in pressure inside the heart. As such, BNP is a commonly assessed marker in the context of heart failure. BNP level are generally higher in subjects with reduced cardiac function relative to subjects with a normal heart. In an example, treatment improves a subjects BNP level. For example, BNP level can be decreased from baseline or the level observed prior to treatment with the methods of the disclosure. In an example, BNP level is reduced to less than 500 pg/ml. In another example, BNP level is reduced to less than 400 pg/ml. In another example, BNP level is reduced to less than 300 pg/ml. In another example, BNP level is reduced to less than 200 pg/ml. In another example, BNP level is reduced to less than 150 pg/ml. In another example, BNP level is reduced to between 100 pg/ml and 400 pg/ml. In another example, BNP level is reduced to between 100 pg/ml and 300 pg/ml.

In an example, treatment improves a subjects left ventricular ejection fraction (LVEF) percentage. For example, LVEF % can be increased to greater than 55%. In an example, LVEF % is increased to greater than 58%. In another example, LVEF % is increased to greater than 60%. In another example, LVEF % is increased to greater than 65%. In an example, the LVEF is measured via echocardiogram.

The term “prevent” or “preventing” as used herein include administering a population of mesenchymal lineage stem or precursor cells and/or progeny thereof and/or soluble factors derived therefrom to thereby stop or inhibit the development of at least one symptom of hyperinflammation.

The term “hyperinflammation” is used in the context of the present disclosure refer to severe and ongoing inflammatory process in body. For example, hyperinflammation can refer to severe and ongoing inflammatory process in airway and/or lungs, kidney or liver. In this way, hyperinflammation can affect multiple organs in the body and their vasculature. In an example, the hyperinflammation is triggered by viral infection. In an example, hyperinflammation is associated with a cytokine storm or cytokine release syndrome (CRS). In an example, the cytokine storm or CRS involves significant release of inflammatory cytokines such as IL-6.

In an example, the hyperinflammation leads to secondary (or acquired) hemophagocytic lymphohistiocytosis (sHLH). Accordingly, in an example, the methods of the present disclosure encompass treatment of hemophagocytic lymphohistiocytosis (sHLH).

In another example, hyperinflammation is associated with elevated CRP, PCT, IL-6 and/or ferritin. For example, ferritin may be greater than 2000 mg/dl. In another example, ferritin is greater than 2500 mg/dl. In an example, hyperinflammation is associated with bacterial infection. In an example, subjects treated according to the present disclosure have elevated circulating CRP levels. For example, subjects treated according to the present disclosure can have circulating CRP levels greater than 100 mg/dl. In another example, treated subjects have circulating CRP levels greater than 120 mg/dl. In another example, treated subjects have circulating CRP levels greater than 150 mg/dl. In another example, treated subjects have circulating CRP levels between 90 mg/dl and 300 mg/dl.

In other examples, hyperinflammation is associated with elevated triglycerides or decreased fibrinogen. For example, subjects treated according to the present disclosure can have triglyceride levels >1.5 mmol/L. In another example, treated subjects have triglyceride levels >2, >3, >4 mmol/L. In another example, treated subjects have triglyceride levels between 1.5 and 5 mmol/L. In another example, treated subjects have fibrinogen levels less than or equal to 2.5 g/L. In another example, treated subjects have fibrinogen levels less than 2.5 g/L.

In an example, the hyperinflammation leads to multi-system inflammatory syndrome (MIS). For example, hyperinflammation can lead to MIS in children (MIS-C).

In an example, hyperinflammation is caused by a viral infection. For example, the hyperinflammation can be caused by a rhinovirus, an influenza virus, a respiratory syncytial virus (RSV) or a coronavirus. In an example, the hyperinflammation can be caused by a coronavirus. For example, the coronavirus can be coronavirus (SARS-CoV), Middle East Respiratory Syndrome coronavirus (MERS-CoV) or COVID-19. In an example, the hyperinflammation is caused by Epstein-Barr virus (EBV) or herpes simplex virus (HSV).

In an example, methods of the present disclosure inhibit disease progression or disease complication in a subject. “Inhibition” of disease progression or disease complication in a subject means preventing or reducing the disease progression and/or disease complication in the subject. Accordingly, in an example, methods of the disclosure inhibit progression of hyperinflammation to sHLH.

The term “subject” as used herein refers to a human subject. For example, the subject can be an adult. In another example, the subject can be a child. In another example, the subject can be an adolescent. Terms such as “subject”, “patient” or “individual” are terms that can, in context, be used interchangeably in the present disclosure.

Subjects treated according to the present disclosure may have symptoms indicative of hyperinflammation. Exemplary symptoms may include fatigue, trouble breathing, shortness of breath, inability or decreased ability to exercise, coughing with or without blood or mucus, pain when breathing in or out, wheezing, chest tightness, unexplained weight loss, and musculoskeletal pain.

In another example, the subject is 18-75 years of age. In an example, the subject is greater than 50 years of age. In another example, the subject has ARDS. In another example, the subject has pneumonia. In an example, the subject is less than 30 years old. In example, the subject is less than 21 years old.

In another example, the subject has ARDS secondary to viral infection. In an example, the subjects ARDS is secondary to infection with a rhinovirus, an influenza virus, a respiratory syncytial virus (RSV) or a coronavirus. In an example, the subjects ARDS is secondary to infection with a coronavirus. For example, the subjects ARDS can be secondary to infection with SARS-CoV, MERS-CoV or COVID-19.

In an example, the methods of the present disclosure prevent or treat subjects with multi-system inflammatory syndrome (MIS). In an example, the subject is a child with MIS. For example, a subject with MIS can be between 1 month and 18 years old. In an example, the subject has acute heart failure. Heart failure (HF) is a clinical syndrome generally characterised by a constellation of symptoms (dyspnoea, orthopnoea, lower limb swelling) and signs (elevated jugular venous pressure, pulmonary congestion). Acute heart failure is broadly defined as a rapid onset of new or worsening signs and symptoms of heart failure. In an example, the subject has depressed left ventricular ejection fraction. For example, the subjects LVEF may be less than 45%. In an example, the LVEF is less than 40%. In another example, the LVEF is less than 30%.

In an example, a subject with MIS meets the following criteria:
One or more of the following:
   Hypotension or shock (cardiogenic or vasogenic)
   Features of severe cardiac illness including but not limited to myocarditis, pericarditis, or valvulitis, significantly elevated troponin/proBNP, or coronary artery abnormalities.
   Other severe end-organ involvement, including but not limited to neurological or renal disease (excluding severe respiratory disease alone);

Or,
Two or more of the following:
   Maculopapular rash
   Bilateral non-purulent conjunctivitis
   Mucocutaneous inflammatory signs (mouth, hands, or feet)
   Acute gastrointestinal symptoms (diarrhea, vomiting, or abdominal pain).

In an example, subjects meeting the above referenced MIS criteria have a fever (temperature greater than or equal to 38 degrees Celsius).

In another example, a subject with MIS presents with elevated inflammatory markers. For example, the subject can present with one or more of neutrophilia, lymphopenia, thrombocytopenia, hypoalbuminemia, elevated CRP, erythrocyte sedimentation rate (ESR), fibrinogen, D-dimer, ferritin, lactic acid dehydrogenase (LDH), interleukin 6 (IL-6), elevated procalcitonin. In an example, the subject has two or more of neutrophilia, lymphopenia, thrombocytopenia, hypoalbuminemia, elevated CRP, ESR, fibrinogen, D-dimer, ferritin, LDH, IL-6, elevated procalcitonin.

In an example, the subject has one or more of myocarditis, pericarditis, or valvulitis. In an example, the subject has viral induced myocarditis, pericarditis, or valvulitis. For example, the subject can have viral myocarditis.

In an example, the MIS is secondary to infection with SARS-CoV, MERS-CoV or COVID-19.

In another example, the methods of the present disclosure prevent or treat subjects with mild ARDS. In another example, the methods of the present disclosure prevent or treat subjects with moderate ARDS. In another example, the methods of the present disclosure prevent or treat subjects with severe ARDS. In another example, the methods of the present disclosure prevent or treat subjects with moderate or severe ARDS. In another example, the methods of the present disclosure prevent or treat subjects with moderate, severe or very severe ARDS.

The term “thrombosis” is used herein to refer to the formation of a thrombus or blood clot. In an example, the thrombosis is “arterial thrombosis” where the blood clot develops in an artery. Such blood clots are particularly dangerous to a subject as they can obstruct blood flow to major organs such as the heart or brain. In an example, the thrombosis is “venous thrombosis” where the blood clot develops in a vein.

The term “thrombolytic” is used to refer to compositions of the disclosure which breakdown blood clots. In an example, thrombolytic compositions of the disclosure reduce a subjects risk of thrombosis. In an example, the subjects risk is reduced relative to a subject who does not receive the composition. In an example, administration of the composition reduces the risk of arterial thrombosis. Accordingly, in an example, administration of the composition reduces the risk of heart attack or stroke.

The term “pulmonary embolism” is used herein to refer to a blockage of an artery in the lungs by a substance that has moved from elsewhere in the body through the bloodstream.

As used herein, the term “genetically unmodified” refers to cells that have not been modified by transfection with a nucleic acid. For the avoidance of doubt, in the context of the present disclosure a mesenchymal lineage precursor or stem cell transfected with a nucleic acid encoding Ang1 would be considered genetically modified.

The term “total dose” is used in the context of the present disclosure to refer to the total number of cells received by the subject treated according to the present disclosure. In an example, the total dose consists of one administration of cells. In another example, the total dose consists of two administrations of cells. In another example, the total dose consists of three administrations of cells. In another example, the total dose consists of four or more administrations of cells. For example, the total dose can consist of two to four administrations of cells.

Mesenchymal Lineage Precursor Cells

As used herein, the term “mesenchymal lineage precursor or stem cell (MLPSC)” refers to undifferentiated multipotent cells that have the capacity to self-renew while maintaining multipotency and the capacity to differentiate into a number of cell types either of mesenchymal origin, for example, osteoblasts, chondrocytes, adipocytes, stromal cells, fibroblasts and tendons, or non-mesodermal origin, for example, hepatocytes, neural cells and epithelial cells. For the avoidance of doubt, a “mesenchymal lineage precursor cell” refers to a cell which can differentiate into a mesenchymal cell such as bone, cartilage, muscle and fat cells, and fibrous connective tissue.

The term “mesenchymal lineage precursor or stem cells” includes both parent cells and their undifferentiated progeny. The term also includes mesenchymal precursor cells, multipotent stromal cells, mesenchymal stem cells (MSCs), perivascular mesenchymal precursor cells, and their undifferentiated progeny.

Mesenchymal lineage precursor or stem cells can be autologous, allogeneic, xenogenic, syngenic or isogenic. Autologous cells are isolated from the same individual to which they will be reimplanted. Allogeneic cells are isolated from a donor of the same species. Xenogenic cells are isolated from a donor of another species. Syngenic or isogenic cells are isolated from genetically identical organisms, such as twins, clones, or highly inbred research animal models.

In an example, the mesenchymal lineage precursor or stem cells are allogeneic. In an example, the allogeneic mesenchymal lineage precursor or stem cells are culture expanded and cryopreserved.

Mesenchymal lineage precursor or stem cells reside primarily in the bone marrow, but have also shown to be present in diverse host tissues including, for example, cord blood and umbilical cord, adult peripheral blood, adipose tissue, trabecular bone and dental pulp. They are also found in skin, spleen, pancreas, brain, kidney, liver, heart, retina, brain, hair follicles, intestine, lung, lymph node, thymus, ligament, tendon, skeletal muscle, dermis, and periosteum; and are capable of differentiating into germ lines such as mesoderm and/or endoderm and/or ectoderm. Thus, mesenchymal lineage precursor or stem cells are capable of differentiating into a large number of cell types including, but not limited to, adipose, osseous, cartilaginous, elastic, muscular, and fibrous connective tissues. The specific lineage-commitment and differentiation pathway which these cells enter depends upon various influences from mechanical influences and/or endogenous bioactive factors, such as growth factors, cytokines, and/or local microenvironmental conditions established by host tissues.

The terms “enriched”, “enrichment” or variations thereof are used herein to describe a population of cells in which the proportion of one particular cell type or the proportion of a number of particular cell types is increased when compared with an untreated population of the cells (e.g., cells in their native environment). In one example, a population enriched for mesenchymal lineage precursor or stem cells comprises at least about 0.1% or 0.5% or 1% or 2% or 5% or 10% or 15% or 20% or 25% or 30% or 50% or 75% mesenchymal lineage precursor or stem cells. In this regard, the term “population of cells enriched for mesenchymal lineage precursor or stem cells” will be taken to provide explicit support for the term “population of cells comprising X % mesenchymal lineage precursor or stem cells”, wherein X % is a percentage as recited herein. The mesenchymal lineage precursor or stem cells can, in some examples, form clonogenic colonies, e.g. CFU-F (fibroblasts) or a subset thereof (e.g., 50% or 60% or 70% or 70% or 90% or 95%) can have this activity.

In an example of the present disclosure, the mesenchymal lineage precursor or stem cells are mesenchymal stem cells (MSCs). The MSCs may be a homogeneous composition or may be a mixed cell population enriched in MSCs. Homogeneous MSC compositions may be obtained by culturing adherent marrow or periosteal cells, and the MSCs may be identified by specific cell surface markers which are identified with unique monoclonal antibodies. A method for obtaining a cell population enriched in MSCs is described, for example, in U.S. Pat. No. 5,486,359. Alternative sources for MSCs include, but are not limited to, blood, skin, cord blood, muscle, fat, bone, and perichondrium. In an example, the MSCs are allogeneic. In an example, the MSCs are cryopreserved. In an example, the MSCs are culture expanded and cryopreserved.

In another example, the mesenchymal lineage precursor or stem cells are CD29+, CD54+, CD73+, CD90+, CD102+, CD105+, CD106+, CD166+, MHC1+MSCs.

Isolated or enriched mesenchymal lineage precursor or stem cells can be expanded in vitro by culture. Isolated or enriched mesenchymal lineage precursor or stem cells can be cryopreserved, thawed and subsequently expanded in vitro by culture.

In one example, isolated or enriched mesenchymal lineage precursor or stem cells are seeded at 50,000 viable cells/cm² in culture medium (serum free or serum-supplemented), for example, alpha minimum essential media (αMEM) supplemented with 5% fetal bovine serum (FBS) and glutamine, and allowed to adhere to the culture vessel overnight at 37° C., 20% O₂. The culture medium is subsequently replaced and/or altered as required and the cells cultured for a further 68 to 72 hours at 37° C., 5% 02.

As will be appreciated by those of skill in the art, cultured mesenchymal lineage precursor or stem cells are phenotypically different to cells in vivo. For example, in one embodiment they express one or more of the following markers, CD44, NG2, DC146 and CD140b. Cultured mesenchymal lineage precursor or stem cells are also biologically different to cells in vivo, having a higher rate of proliferation compared to the largely non-cycling (quiescent) cells in vivo.

In one example, the population of cells is enriched from a cell preparation comprising STRO-1+ cells in a selectable form. In this regard, the term “selectable form” will be understood to mean that the cells express a marker (e.g., a cell surface marker) permitting selection of the STRO-1+ cells. The marker can be STRO-1, but need not be. For example, as described and/or exemplified herein, cells (e.g., mesenchymal precursor cells) expressing STRO-2 and/or STRO-3 (TNAP) and/or STRO-4 and/or VCAM-1 and/or CD146 and/or 3G5 also express STRO-1 (and can be STRO-1bright). Accordingly, an indication that cells are STRO-1+ does not mean that the cells are selected solely by STRO-1 expression. In one example, the cells are selected based on at least STRO-3 expression, e.g., they are STRO-3+(TNAP+).

Reference to selection of a cell or population thereof does not necessarily require selection from a specific tissue source. As described herein STRO-1+ cells can be selected from or isolated from or enriched from a large variety of sources. That said, in some examples, these terms provide support for selection from any tissue comprising STRO-1+ cells (e.g., mesenchymal precursor cells) or vascularized tissue or tissue comprising pericytes (e.g., STRO-1+ pericytes) or any one or more of the tissues recited herein.

In one example, the cells used in the present disclosure express one or more markers individually or collectively selected from the group consisting of TNAP+, VCAM-1+, THY-1+, STRO-2+, STRO-4+(HSP-9013), CD45+, CD146+, 3G5+ or any combination thereof.

By “individually” is meant that the disclosure encompasses the recited markers or groups of markers separately, and that, notwithstanding that individual markers or groups of markers may not be separately listed herein the accompanying claims may define such marker or groups of markers separately and divisibly from each other.

By “collectively” is meant that the disclosure encompasses any number or combination of the recited markers or groups of markers, and that, notwithstanding that such numbers or combinations of markers or groups of markers may not be specifically listed herein the accompanying claims may define such combinations or sub-combinations separately and divisibly from any other combination of markers or groups of markers.

As used herein the term “TNAP” is intended to encompass all isoforms of tissue non-specific alkaline phosphatase. For example, the term encompasses the liver isoform (LAP), the bone isoform (BAP) and the kidney isoform (KAP). In one example, the TNAP is BAP. In one example, TNAP as used herein refers to a molecule which can bind the STRO-3 antibody produced by the hybridoma cell line deposited with ATCC on 19 Dec. 2005 under the provisions of the Budapest Treaty under deposit accession number PTA-7282.

Furthermore, in one example, the STRO-1+ cells are capable of giving rise to clonogenic CFU-F.

In one example, a significant proportion of the STRO-1+ cells are capable of differentiation into at least two different germ lines. Non-limiting examples of the lineages to which the STRO-1+ cells may be committed include bone precursor cells; hepatocyte progenitors, which are multipotent for bile duct epithelial cells and hepatocytes; neural restricted cells, which can generate glial cell precursors that progress to oligodendrocytes and astrocytes; neuronal precursors that progress to neurons; precursors for cardiac muscle and cardiomyocytes, glucose-responsive insulin secreting pancreatic beta cell lines. Other lineages include, but are not limited to, odontoblasts, dentin-producing cells and chondrocytes, and precursor cells of the following: retinal pigment epithelial cells, fibroblasts, skin cells such as keratinocytes, dendritic cells, hair follicle cells, renal duct epithelial cells, smooth and skeletal muscle cells, testicular progenitors, vascular endothelial cells, tendon, ligament, cartilage, adipocyte, fibroblast, marrow stroma, cardiac muscle, smooth muscle, skeletal muscle, pericyte, vascular, epithelial, glial, neuronal, astrocyte and oligodendrocyte cells.

In an example, mesenchymal lineage precursor or stem cells are obtained from a single donor, or multiple donors where the donor samples or mesenchymal lineage precursor or stem cells are subsequently pooled and then culture expanded.

Mesenchymal lineage precursor or stem cells encompassed by the present disclosure may also be cryopreserved prior to administration to a subject. In an example, mesenchymal lineage precursor or stem cells are culture expanded and cryopreserved prior to administration to a subject.

In an example, the present disclosure encompasses mesenchymal lineage precursor or stem cells as well as progeny thereof, soluble factors derived therefrom, and/or extracellular vesicles isolated therefrom. In another example, the present disclosure encompasses mesenchymal lineage precursor or stem cells as well as extracellular vesicles isolated therefrom. For example, it is possible to culture expand mesenchymal precursor lineage or stem cells of the disclosure for a period of time and under conditions suitable for secretion of extracellular vesicles into the cell culture medium. Secreted extracellular vesicles can subsequently be obtained from the culture medium for use in therapy.

The term “extracellular vesicles” as used herein, refers to lipid particles naturally released from cells and ranging in size from about 30 nm to as a large as 10 microns, although typically they are less than 200 nm in size. They can contain proteins, nucleic acids, lipids, metabolites, or organelles from the releasing cells (e.g., mesenchymal stem cells; STRO-1±cells).

The term “exosomes” as used herein, refers to a type of extracellular vesicle generally ranging in size from about 30 nm to about 150 nm and originating in the endosomal compartment of mammalian cells from which they are trafficked to the cell membrane and released. They may contain nucleic acids (e.g., RNA; microRNAs), proteins, lipids, and metabolites and function in intercellular communication by being secreted from one cell and taken up by other cells to deliver their cargo.

Culture Expansion of the Cells

In an example, mesenchymal lineage precursor or stem cells are culture expanded. “Culture expanded” mesenchymal lineage precursor or stem cells media are distinguished from freshly isolated cells in that they have been cultured in cell culture medium and passaged (i.e. sub-cultured). In an example, culture expanded mesenchymal lineage precursor or stem cells are culture expanded for about 4-10 passages. In an example, mesenchymal lineage precursor or stem cells are culture expanded for at least 5, at least 6, at least 7, at least 8, at least 9, at least 10 passages. For example, mesenchymal lineage precursor or stem cells can be culture expanded for at least 5 passages. In an example, mesenchymal lineage precursor or stem cells can be culture expanded for at least 5-10 passages. In an example, mesenchymal lineage precursor or stem cells can be culture expanded for at least 5-8 passages. In an example, mesenchymal lineage precursor or stem cells can be culture expanded for at least 5-7 passages. In an example, mesenchymal lineage precursor or stem cells can be culture expanded for more than 10 passages. In another example, mesenchymal lineage precursor or stem cells can be culture expanded for more than 7 passages. In these examples, stem cells may be culture expanded before being cryopreserved to provide an intermediate cryopreserved MLPSC population. In an example, compositions of the disclosure are prepared from an intermediate cryopreserved MLPSC population. For example, an intermediate cryopreserved MLPSC population can be further culture expanded prior to administration as is discussed further below. Accordingly, in an example, mesenchymal lineage precursor or stem cells are culture expanded and cryopreserved. In an embodiment of these examples, mesenchymal lineage precursor or stem cells can be obtained from a single donor, or multiple donors where the donor samples or mesenchymal lineage precursor or stem cells are subsequently pooled and then culture expanded. In an example, the culture expansion process comprises:
i. expanding by passage expansion the number of viable cells to provide a preparation of at least about 1 billion of the viable cells, wherein the passage expansion comprises establishing a primary culture of isolated mesenchymal lineage precursor or stem cells and then serially establishing a first non-primary (P1) culture of isolated mesenchymal lineage precursor or stem cells from the previous culture;
ii. expanding by passage expansion the P1 culture of isolated mesenchymal lineage precursor or stem cells to a second non-primary (P2) culture of mesenchymal lineage precursor or stem cells; and,
iii. preparing and cryopreserving an in-process intermediate mesenchymal lineage precursor or stem cells preparation obtained from the P2 culture of mesenchymal lineage precursor or stem cells; and,
iv. thawing the cryopreserved in-process intermediate mesenchymal lineage precursor or stem cells preparation and expanding by passage expansion the in-process intermediate mesenchymal lineage precursor or stem cells preparation.

In an example, the expanded mesenchymal lineage precursor or stem cell preparation has an antigen profile and an activity profile comprising:
i. less than about 0.75% CD45+ cells;
ii. at least about 95% CD105+ cells;
iii. at least about 95% CD166+ cells.

In an example, the expanded mesenchymal lineage precursor or stem cell preparation is capable of inhibiting IL2Ra expression by CD3/CD28-activated PBMCs by at least about 30% relative to a control.

In an example, culture expanded mesenchymal lineage precursor or stem cells are culture expanded for about 4-10 passages, wherein the mesenchymal lineage precursor or stem cells have been cryopreserved after at least 2 or 3 passages before being further culture expanded. In an example, mesenchymal lineage precursor or stem cells are culture expanded for at least 1, at least 2, at least 3, at least 4, at least 5 passages, cryopreserved and then further culture expanded for at least 1, at least 2, at least 3, at least 4, at least 5 passages before being administered or further cryopreserved.

In an example, the majority of mesenchymal lineage precursor or stem cells in compositions of the disclosure are of about the same generation number (i.e., they are within about 1 or about 2 or about 3 or about 4 cell doublings of each other). In an example, the average number of cell doublings in the present compositions is about 20 to about 25 doublings. In an example, the average number of cell doublings in the present compositions is about 9 to about 13 (e.g., about 11 or about 11.2) doublings arising from the primary culture, plus about 1, about 2, about 3, or about 4 doublings per passage (for example, about 2.5 doublings per passage). Exemplary average cell doublings in present compositions are any of about 13.5, about 16, about 18.5, about 21, about 23.5, about 26, about 28.5, about 31, about 33.5, and about 36 when produced by about 1, about 2, about 3, about 4, about 5, about 6, about 7, about 8, about 9, and about 10 passages, respectively.

The process of mesenchymal lineage precursor or stem cell isolation and ex vivo expansion can be performed using any equipment and cell handing methods known in the art. Various culture expansion embodiments of the present disclosure employ steps that require manipulation of cells, for example, steps of seeding, feeding, dissociating an adherent culture, or washing. Any step of manipulating cells has the potential to insult the cells. Although mesenchymal lineage precursor or stem cells can generally withstand a certain amount of insult during preparation, cells are preferably manipulated by handling procedures and/or equipment that adequately performs the given step(s) while minimizing insult to the cells.

In an example, mesenchymal lineage precursor or stem cells are washed in an apparatus that includes a cell source bag, a wash solution bag, a recirculation wash bag, a spinning membrane filter having inlet and outlet ports, a filtrate bag, a mixing zone, an end product bag for the washed cells, and appropriate tubing, for example, as described in U.S. Pat. No. 6,251,295, which is hereby incorporated by reference.

In an example, a mesenchymal lineage precursor or stem cell composition according to the present disclosure is 95% homogeneous with respect to being CD105 positive and CD166 positive and being CD45 negative. In an example, this homogeneity persists through ex vivo expansion; i.e. though multiple population doublings. In an example, the composition comprises at least one therapeutic dose of mesenchymal lineage precursor or stem cells and the mesenchymal lineage precursor or stem cells comprise less than about 1.25% CD45+ cells, at least about 95% CD105+ cells, and at least about 95% CD166+ cells. In an example, this homogeneity persists after cryogenic storage and thawing, where the cells also generally have a viability of about 70% or more.

In an example, compositions of the disclosure comprise mesenchymal lineage precursor or stem cells which express substantial levels of TNFR1, for example greater than 13 pg of TNFR1 per million mesenchymal lineage precursor or stem cells. In an example, this phenotype is stable throughout ex vivo expansion and cryogenic storage. In an example, expression of levels of TNFR1 in the range of about 13 to about 179 pg (e.g. about 13 pg to about 44 pg) per million mesenchymal lineage precursor or stem cells is associated with a desirous therapeutic potential which also persists through ex vivo expansion and cryopreservation.

In an example, the culture expanded mesenchymal lineage precursor or stem cells express Tumor necrosis factor receptor 1 (TNFR1) in an amount of at least 110 pg/ml. For example, the mesenchymal lineage precursor or stem cells can express TNFR1 in an amount of at least 150 pg/ml, or at least 200 pg/ml, or at least 250 pg/ml, or at least 300 pg/ml, or at least 320 pg/ml, or at least 330 pg/ml, or at least 340 pg/ml, or at least 350 pg/ml.

In an example, the mesenchymal lineage precursor or stem cells express TNFR1 in an amount of at least 13 pg/10⁶ cells. For example, the mesenchymal lineage precursor or stem cells express TNFR1 in an amount of at least 15 pg/10⁶ cells, or at least 20 pg/10⁶ cells, or at least 25 pg/10⁶ cells, or at least 30 pg/10⁶ cells, or at least 35 pg/10⁶ cells, or at least 40 pg/10⁶ cells, or at least 45 pg/10⁶ cells, or at least 50 pg/10⁶ cells.

In another example, mesenchymal lineage precursor or stem cells disclosed herein inhibit IL-2Ra expression on T-cells. In an example, mesenchymal lineage precursor or stem cells can inhibit IL-2Ra expression by at least about 30%, alternatively at least about 35%, alternatively at least about 40%, alternatively at least about 45%, alternatively at least about 50%, alternatively at least about 55%, alternatively at least about 60.

In an example, compositions of the disclosure comprise at least one therapeutic dose of mesenchymal lineage precursor or stem cells which, for example, can comprise at least about 100 million cells or about 125 million cells.

Modification of the Cells

In an example, mesenchymal lineage precursor or stem cells of the present disclosure may be altered in such a way that upon administration, lysis of the cell is inhibited. Alteration of an antigen can induce immunological non-responsiveness or tolerance, thereby preventing the induction of the effector phases of an immune response (e.g., cytotoxic T cell generation, antibody production etc.) which are ultimately responsible for rejection of foreign cells in a normal immune response. Antigens that can be altered to achieve this goal include, for example, MHC class I antigens, MHC class II antigens, LFA-3 and ICAM-1.

The mesenchymal lineage precursor or stem cells may also be genetically modified to express proteins of importance for the differentiation and/or maintenance of striated skeletal muscle cells. Exemplary proteins include growth factors (TGF-β, insulin-like growth factor 1 (IGF-1), FGF), myogenic factors (e.g. myoD, myogenin, myogenic factor 5 (Myf5), myogenic regulatory factor (MRF)), transcription factors (e.g. GATA-4), cytokines (e.g. cardiotropin-1), members of the neuregulin family (e.g. neuregulin 1, 2 and 3) and homeobox genes (e.g. Csx, tinman and NKx family).

Mesenchymal lineage precursor or stem cells of the disclosure can also be modified to carry or express an anti-viral agent or a thrombolytic agent. In an example, the agent is an anti-viral drug. In an example, the agent is anti-influenza. In an example, the agent is anti-SARS-CoV (e.g. SARS-Cov2). An exemplary agent is remdesivir. In an example, the agent is a thrombolytic drug. Examples of thrombolytic agents include Eminase (anistreplase), Retavase (reteplase), Streptase (streptokinase, kabikinase). In an example, the thrombolytic agent is heparin.

Mesenchymal precursor or stem cells of the disclosure may be modified to carry an anti-viral or thrombolytic agent by culturing cells with the agent for a time and under conditions sufficient to allow the agent to be absorbed by the cells. In an example, the anti-viral or thrombolytic agent is added to the culture media of mesenchymal lineage precursor or stem cells disclosed herein. For example, mesenchymal lineage precursor or stem cells disclosed herein can be culture expanded in culture media comprising an anti-viral or thrombolytic agent.

In another example, the anti-viral or thrombolytic agent is a peptide. In an example, mesenchymal lineage precursor or stem cells are genetically modified to express a an anti-viral or thrombolytic peptide or a nucleic acid encoding the same. In an example, mesenchymal lineage precursor or stem cells are modified via contact with a viral vector in vitro. For example, virus can be added to cell culture medium. Non-viral methods of genetic modification may also be employed. Examples include plasmid transfer and the application of targeted gene integration through the use of integrase or transposase technologies, liposome or protein transduction domain mediated delivery and physical methods such as electroporation.

Efficiencies of genetic modification are rarely 100%, and it is usually desirable to enrich the population for cells that have been successfully modified. In an example, modified cells can be enriched by taking advantage of a functional feature of the new genotype. One exemplary method of enriching modified cells is positive selection using a selectable or screenable marker gene. “Marker gene” refers to a gene that imparts a distinct phenotype to cells expressing the marker gene and thus, allows such transformed cells to be distinguished from cells that do not have the marker. A selectable marker gene confers a trait for which one can “select” based on resistance to a selective agent (e.g., an antibiotic). A screenable marker gene (or reporter gene) confers a trait that one can identify through observation or testing, that is, by “screening” (e.g., β-glucuronidase, luciferase, GFP or other enzyme activity not present in untransformed cells). In an example, genetically modified mesenchymal lineage precursor or stem cells are selected based on resistance to a drug such as neomycin or colorimetric selection based on expression of lacZ.

Compositions of the Disclosure

In one example of the present disclosure the mesenchymal lineage precursor or stem cells and/or progeny thereof and/or soluble factor derived therefrom are administered in the form of a composition. In one example, such a composition comprises a pharmaceutically acceptable carrier and/or excipient. Accordingly, in an example, compositions of the disclosure can comprise culture expanded mesenchymal lineage precursor or stem cells.

The terms “carrier” and “excipient” refer to compositions of matter that are conventionally used in the art to facilitate the storage, administration, and/or the biological activity of an active compound (see, e.g., Remington's Pharmaceutical Sciences, 16th Ed., Mac Publishing Company (1980). A carrier may also reduce any undesirable side effects of the active compound. A suitable carrier is, for example, stable, e.g., incapable of reacting with other ingredients in the carrier. In one example, the carrier does not produce significant local or systemic adverse effect in recipients at the dosages and concentrations employed for treatment.

Suitable carriers for the present disclosure include those conventionally used, e.g., water, saline, aqueous dextrose, lactose, Ringer's solution, a buffered solution, hyaluronan and glycols are exemplary liquid carriers, particularly (when isotonic) for solutions. Suitable pharmaceutical carriers and excipients include starch, cellulose, glucose, lactose, sucrose, gelatin, malt, rice, flour, chalk, silica gel, magnesium stearate, sodium stearate, glycerol monostearate, sodium chloride, glycerol, propylene glycol, water, ethanol, and the like.

In another example, a carrier is a media composition, e.g., in which a cell is grown or suspended. For example, such a media composition does not induce any adverse effects in a subject to whom it is administered.

Exemplary carriers and excipients do not adversely affect the viability of a cell and/or the ability of a cell to reduce, prevent or delay metabolic syndrome and/or obesity.

In one example, the carrier or excipient provides a buffering activity to maintain the cells and/or soluble factors at a suitable pH to thereby exert a biological activity, e.g., the carrier or excipient is phosphate buffered saline (PBS). PBS represents an attractive carrier or excipient because it interacts with cells and factors minimally and permits rapid release of the cells and factors, in such a case, the composition of the disclosure may be produced as a liquid for direct application to the blood stream or into a tissue or a region surrounding or adjacent to a tissue, e.g., by injection.

The mesenchymal lineage precursor or stem cells and/or progeny thereof and/or soluble factor derived therefrom can also be incorporated or embedded within scaffolds that are recipient-compatible and which degrade into products that are not harmful to the recipient. These scaffolds provide support and protection for cells that are to be transplanted into the recipient subjects. Natural and/or synthetic biodegradable scaffolds are examples of such scaffolds.

A variety of different scaffolds may be used successfully in the practice of the disclosure. Exemplary scaffolds include, but are not limited to biological, degradable scaffolds. Natural biodegradable scaffolds include collagen, fibronectin, and laminin scaffolds. Suitable synthetic material for a cell transplantation scaffold should be able to support extensive cell growth and cell function. Such scaffolds may also be resorbable. Suitable scaffolds include polyglycolic acid scaffolds, (e.g., as described by Vacanti, et al. J. Ped. Surg. 23:3-9 1988; Cima, et al. Biotechnol. Bioeng. 38:145 1991; Vacanti, et al. Plast. Reconstr. Surg. 88:753-9 1991); or synthetic polymers such as polyanhydrides, polyorthoesters, and polylactic acid.

In another example, the mesenchymal lineage precursor or stem cells and/or progeny thereof and/or soluble factor derived therefrom may be administered in a gel scaffold (such as Gelfoam from Upjohn Company).

The compositions described herein may be administered alone or as admixtures with other cells. The cells of different types may be admixed with a composition of the disclosure immediately or shortly prior to administration, or they may be co-cultured together for a period of time prior to administration.

In one example, the composition comprises an effective amount or a therapeutically or prophylactically effective amount of mesenchymal lineage precursor or stem cells and/or progeny thereof and/or soluble factor derived therefrom. For example, the composition comprises about 1×10⁵ stem cells to about 1×10⁹ stem cells or about 1.25×10³ stem cells to about 1.25×10⁷ stem cells/kg (80 kg subject). In an example, the composition comprises 2×10⁶ cells/kg. The exact amount of cells to be administered is dependent upon a variety of factors, including the age, weight, and sex of the subject, and the extent and severity of the disorder being treated.

In an example, 50×10⁶ to 200×10⁷ cells are administered. In other examples, 60×10⁶ to 200×10⁶ cells or 75×10⁶ to 150×10⁶ cells are administered. In an example, 75×10⁶ cells are administered. In another example, 150×10⁶ cells are administered.

In an example, the composition comprises greater than 5.00×10⁶ viable cells/mL. In another example, the composition comprises greater than 5.50×10⁶ viable cells/mL. In another example, the composition comprises greater than 6.00×10⁶ viable cells/mL. In another example, the composition comprises greater than 6.50×10⁶ viable cells/mL. In another example, the composition comprises greater than 6.68×10⁶ viable cells/mL.

In an example, the methods of the present disclosure encompass administering a total dose of 600 million cells. For example, a subject treated according to the present disclosure can receive multiple doses of an above referenced composition so long as the total dose of cells does not exceed 600 million cells. For example, the subject may receive 3 doses of 200 million cells. In an example, the total dose of cells is 500 million cells. In an example, the total dose of cells is 400 million cells. For example, the subject may receive 4 doses of 100 million cells. In an example, the subject receives 1 dose of 100 million cells at baseline followed by three doses of 100 million cells administered one per month over three months. In an example, a dose is 2×10⁶ cells/kg. In an example, a dose is 2×10⁶ cells/kg and the subject receives 2 doses or 3 doses. In an example, a dose is 2×10⁶ cells/kg and the subject receives more than 3 doses.

In an example, the mesenchymal lineage precursor or stem cells comprise at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99% of the cell population of the composition.

Compositions of the disclosure may be cryopreserved. Cryopreservation of mesenchymal lineage precursor or stem cells can be carried out using slow-rate cooling methods or ‘fast’ freezing protocols known in the art. Preferably, the method of cryopreservation maintains similar phenotypes, cell surface markers and growth rates of cryopreserved cells in comparison with unfrozen cells.

The cryopreserved composition may comprise a cryopreservation solution. The pH of the cryopreservation solution is typically 6.5 to 8, preferably 7.4.

The cryopreservation solution may comprise a sterile, non-pyrogenic isotonic solution such as, for example, PlasmaLyte A™. 100 mL of PlasmaLyte A™ contains 526 mg of sodium chloride, USP (NaCl); 502 mg of sodium gluconate (C₆H₁₁NaO₇); 368 mg of sodium acetate trihydrate, USP (C₂H₃NaO₂.3H₂O); 37 mg of potassium chloride, USP (KCl); and 30 mg of magnesium chloride, USP (MgCl₂.6H₂O). It contains no antimicrobial agents. The pH is adjusted with sodium hydroxide. The pH is 7.4 (6.5 to 8.0).

The cryopreservation solution may comprise Profreeze™. The cryopreservation solution may additionally or alternatively comprise culture medium, for example, αMEM.

To facilitate freezing, a cryoprotectant such as, for example, dimethylsulfoxide (DMSO), is usually added to the cryopreservation solution. Ideally, the cryoprotectant should be nontoxic for cells and patients, nonantigenic, chemically inert, provide high survival rate after thawing and allow transplantation without washing. However, the most commonly used cryoprotector, DMSO, shows some cytotoxicity. Hydroxylethyl starch (HES) may be used as a substitute or in combination with DMSO to reduce cytotoxicity of the cryopreservation solution.

The cryopreservation solution may comprise one or more of DMSO, hydroxyethyl starch, human serum components and other protein bulking agents. In one example, the cryopreserved solution comprises about 5% human serum albumin (HSA) and about 10% DMSO. The cryopreservation solution may further comprise one or more of methycellulose, polyvinyl pyrrolidone (PVP) and trehalose.

In one embodiment, cells are suspended in 42.5% Profreeze™/50% αMEM/7.5% DMSO and cooled in a controlled-rate freezer.

The cryopreserved composition may be thawed and administered directly to the subject or added to another solution, for example, comprising HA. Alternatively, the cryopreserved composition may be thawed and the mesenchymal lineage precursor or stem cells resuspended in an alternate carrier prior to administration.

In an example, cellular compositions of the disclosure can comprise Plasma-Lyte A, dimethyl sulfoxide (DMSO) and human serum albumin (HSA). For example, compositions of the disclosure may comprise Plasma-Lyte A (70%), DMSO (10%), HSA (25%) solution, the HSA solution comprising 5% HSA and 15% buffer.

In an example, the compositions described herein may be administered as a single dose.

In some examples, the compositions described herein may be administered over multiple doses. For example, at least 2, at least 3, at least 4 doses. In other examples, compositions described herein may be administered over at least 5, at least 6, at least 7, at least 8, at least 9, at least 10 doses.

In one example, the mesenchymal lineage precursor or stem cells can be culture expanded prior to administration to a subject. Various methods of cell culture are known in the art. In an example, mesenchymal lineage precursor or stem cells are culture expanded for about 4-10 passages. In an example, mesenchymal lineage precursor or stem cells are culture expanded for at least 4, at least 5, at least 6, at least 7, at least 8, at least 9, at least 10 passages. In an example, mesenchymal lineage precursor or stem cells are culture expanded for at least 5 passages. In these examples, stem cells may be culture expanded before being cryopreserved.

In an example, mesenchymal lineage precursor or stem cells are culture expanded in a serum free medium prior to administration.

In some examples, the cells are contained within a chamber that does not permit the cells to exit into a subject's circulation but permits factors secreted by the cells to enter the circulation. In this manner soluble factors may be administered to a subject by permitting the cells to secrete the factors into the subject's circulation. Such a chamber may equally be implanted at a site in a subject to increase local levels of the soluble factors.

In an example, mesenchymal lineage precursor or stem cells may be administered systemically. In an example, mesenchymal lineage precursor or stem cells may be administered to the subjects airway. In an example, mesenchymal lineage precursor or stem cells may be administered to the lung(s) of a subject. In another example, compositions of the disclosure are administered intravenously. In another example, compositions are administered intravenously and to the subjects airway.

In an example, mesenchymal lineage precursor or stem cells are administered once weekly. For example, mesenchymal lineage precursor or stem cells can be administered once weekly every two weeks. In another example, mesenchymal lineage precursor or stem cells are administered twice weekly. In an example, mesenchymal lineage precursor or stem cells can be administered once monthly. In an example, two doses of mesenchymal lineage precursor or stem cells are administered once weekly over two weeks. In another example, two doses of mesenchymal lineage precursor or stem cells are administered once weekly every two weeks. In another example, four doses of mesenchymal lineage precursor or stem cells are administered over two weeks before subsequent doses are administered monthly. In an example, two doses of mesenchymal lineage precursor or stem cells can be administered once weekly every two weeks before subsequent doses are administered once monthly. In an example, four doses are administered monthly.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

The following specific examples are to be construed as merely illustrative, and not limitative of the remainder of the disclosure in any way whatsoever. Without further elaboration, it is believed that one skilled in the art can, based on the description herein, utilize the present invention to its fullest extent.

### Examples

### Ex-Vivo Culture-Expanded Adult Allogeneic Bone Marrow Derived Mesenchymal Stem Cells (MSCs), for the Treatment of Hyperinflammation

Composition

The composition is comprised of culture-expanded mesenchymal stromal cells (ceMSC) isolated from the bone marrow of healthy adult donors. The final composition comprises ceMSC formulated in Plasma-Lyte A, dimethyl sulfoxide (DMSO) and human serum albumin (HSA).

Objectives

To determine:
Safety
Change from Baseline in the following:
   White blood cell count.
   P/F ratio.
   circulating CRP levels.
   Ferritin levels

Measurements

Baseline measurements were considered to be those taken prior to administration of first dose (Study Day 0). White blood cell count, P/F ratio, circulating CRP levels and Ferritin levels were measured daily.

Subjects

Patients (n=5) characterized as having moderate COVID-19 related ARDS received intravenous infusion(s) of mesenchymal stem cells (2 million cells per kg).

Analysis

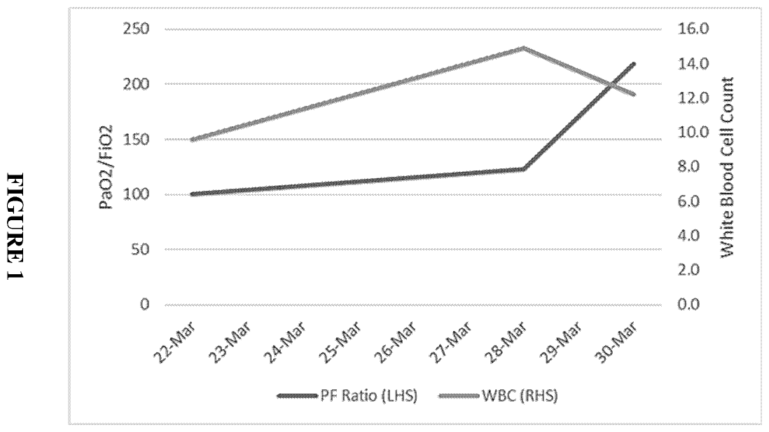

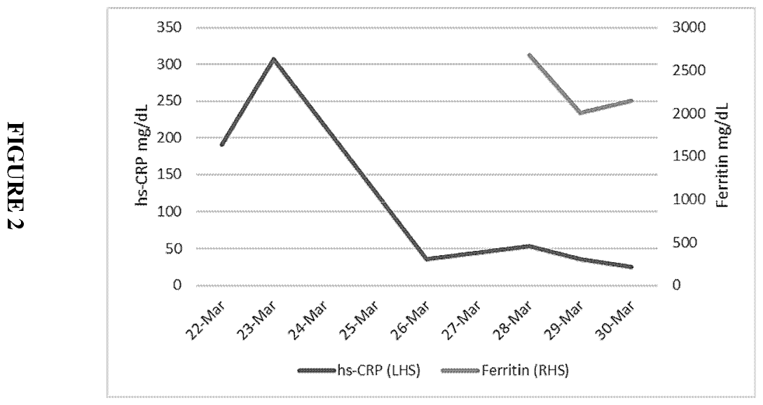

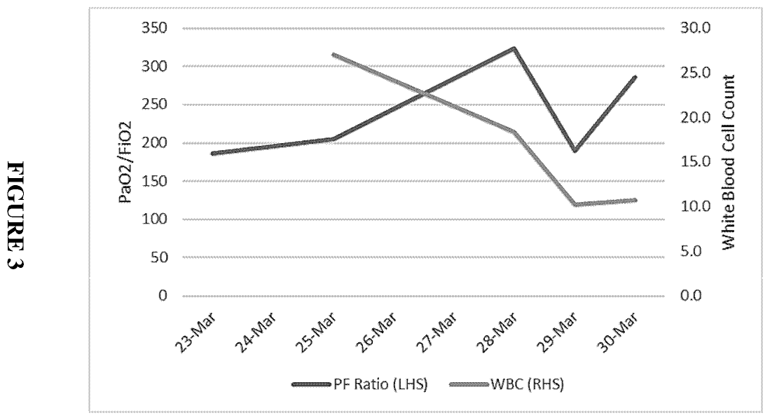

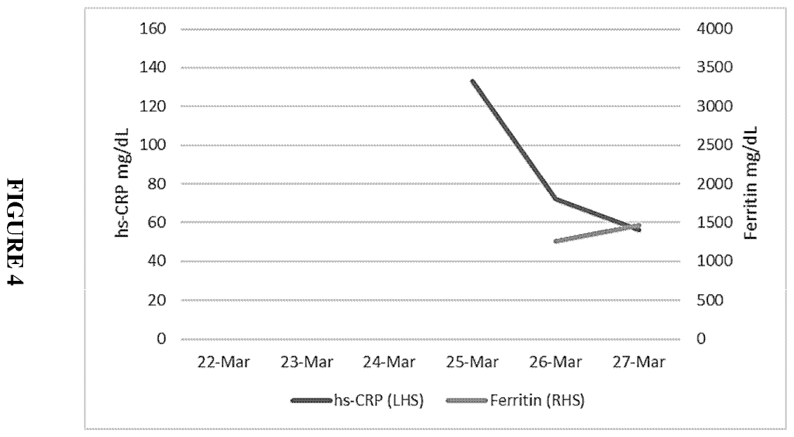

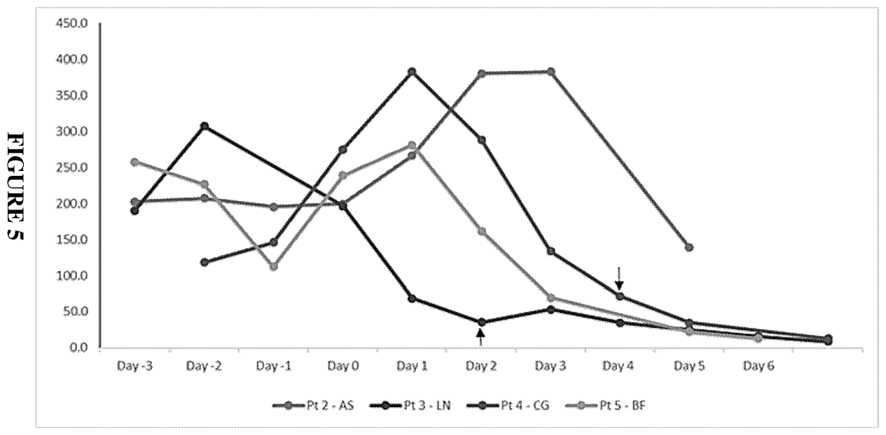

Preliminary data for treated patients is shown in Table 1 and FIGS. 1 to 5.

TABLE 1

| | Age | PF Ratio | WBC | FiO2 | hsCRP | Creatinine | FERR |
|---|---|---|---|---|---|---|---|
| Patient 1 | 60 | ↑ | ↓ | ↓ | ↓↓ | ↓ | ↓ |
| Patient 2 | 39 | ↑↑↑ | ↓↓↓ | ↓↓ | ↓ | ↔ | |
| Patient 3 | 35 | ↑↑ | ↓↓↓ | ↓↓ | ↓↓ | ↔ | ↓↓ |
| Patient 4 | 60 | ↑↑ | ↓ | ↓↓ | | ↔ | |
| Patient 5 | 51 | ↑↑↑ | | ↓ | ↓↓ | ↑ | |

Scale:
↑/↓ Some improvement observed
↑↑/↓↓ More than 50% improvement observed
↑↑↑/↓↓↓ Return to normal range

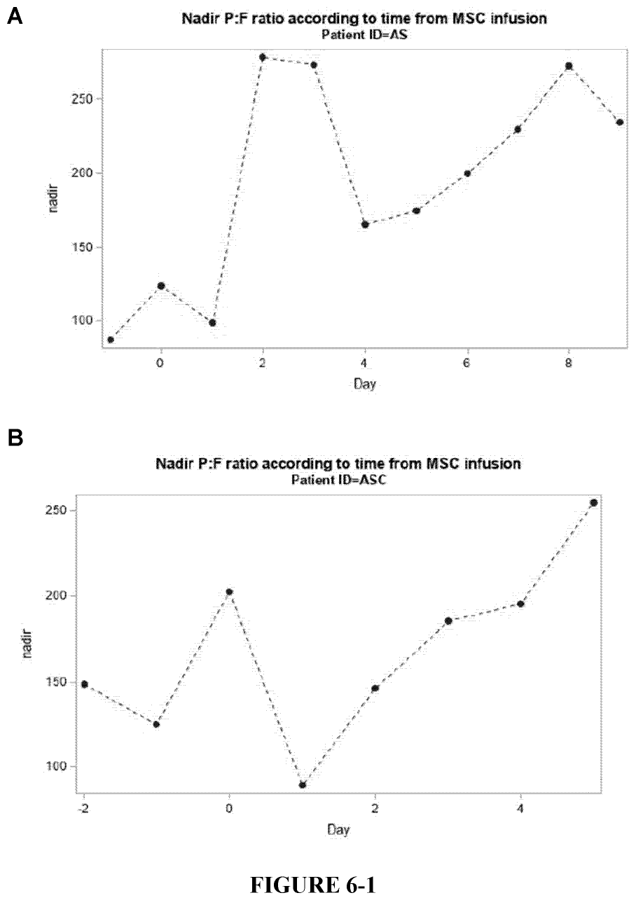

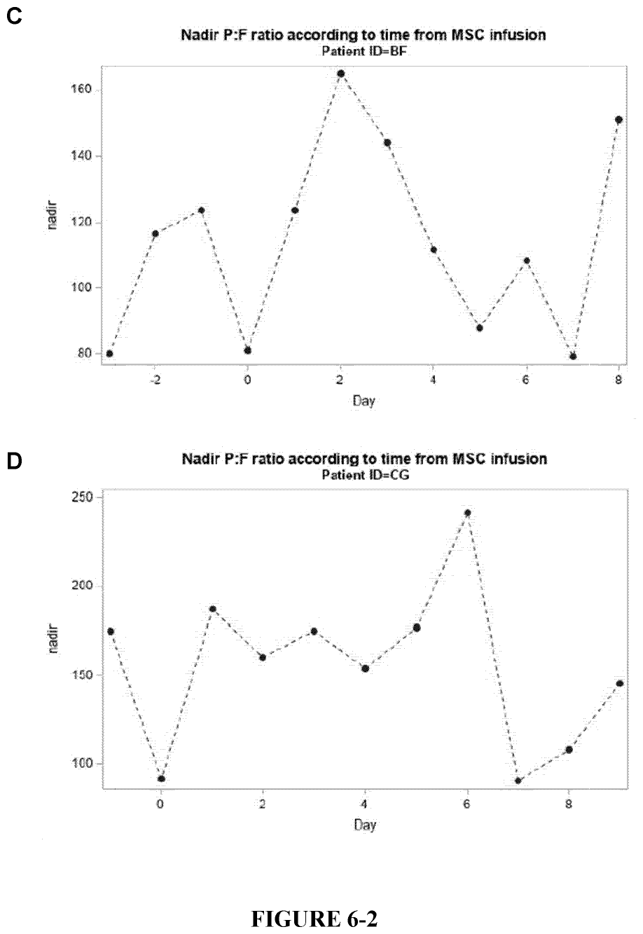

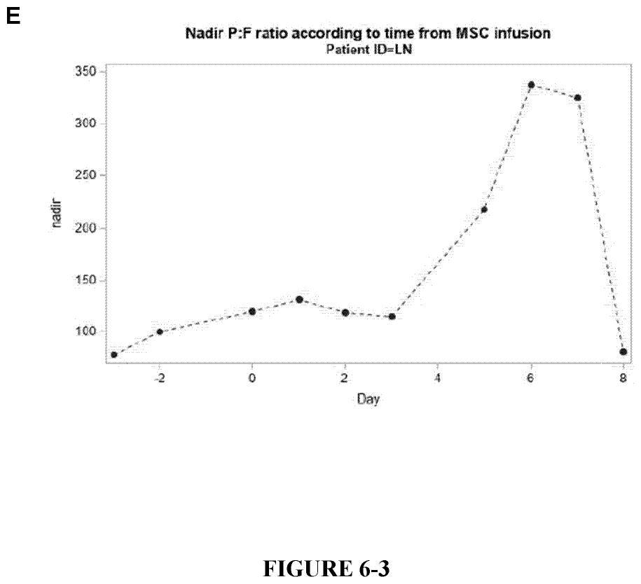

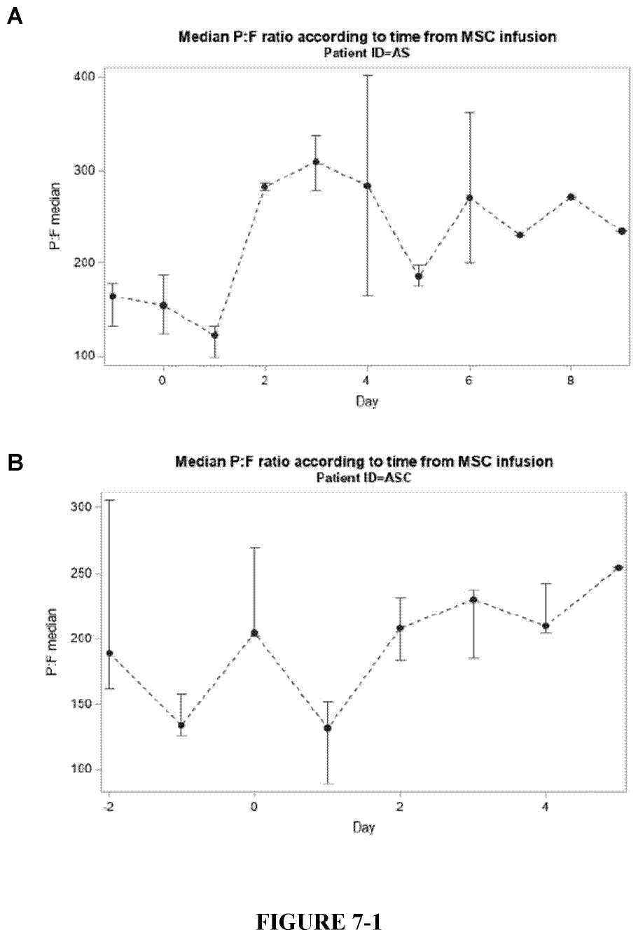

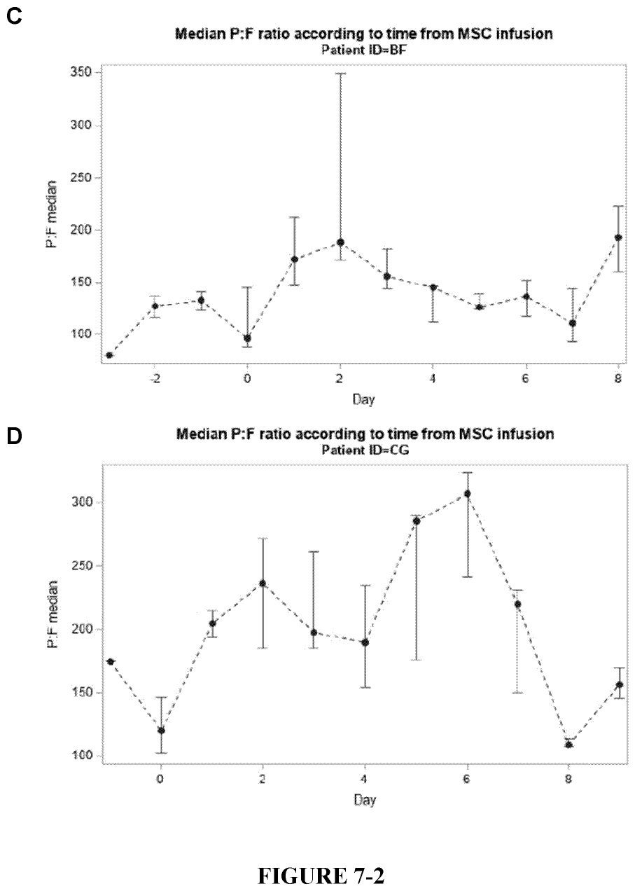

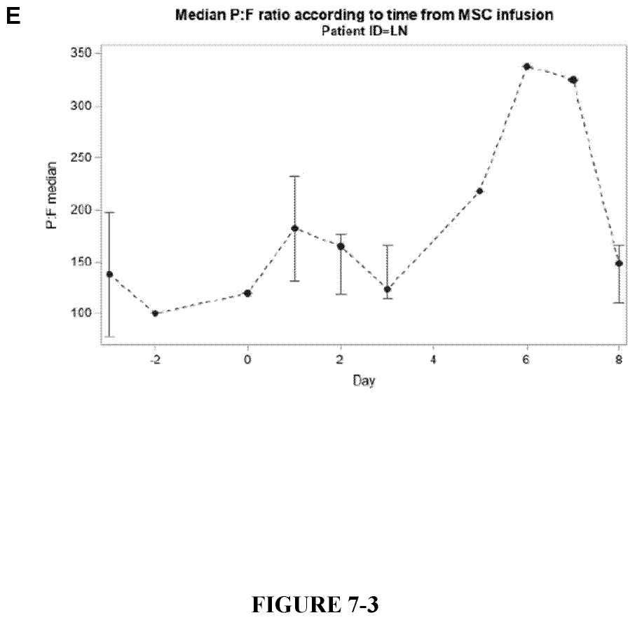

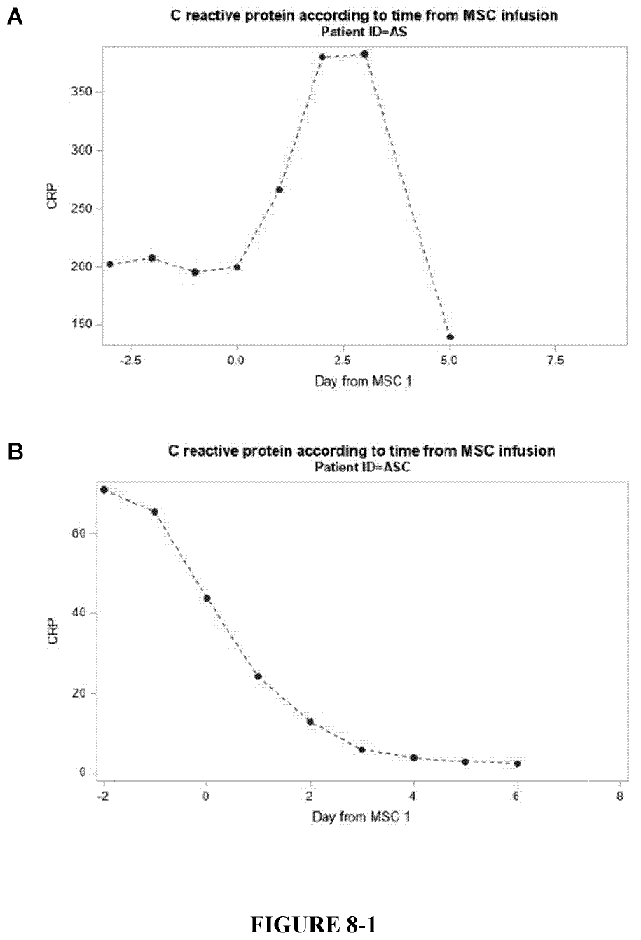

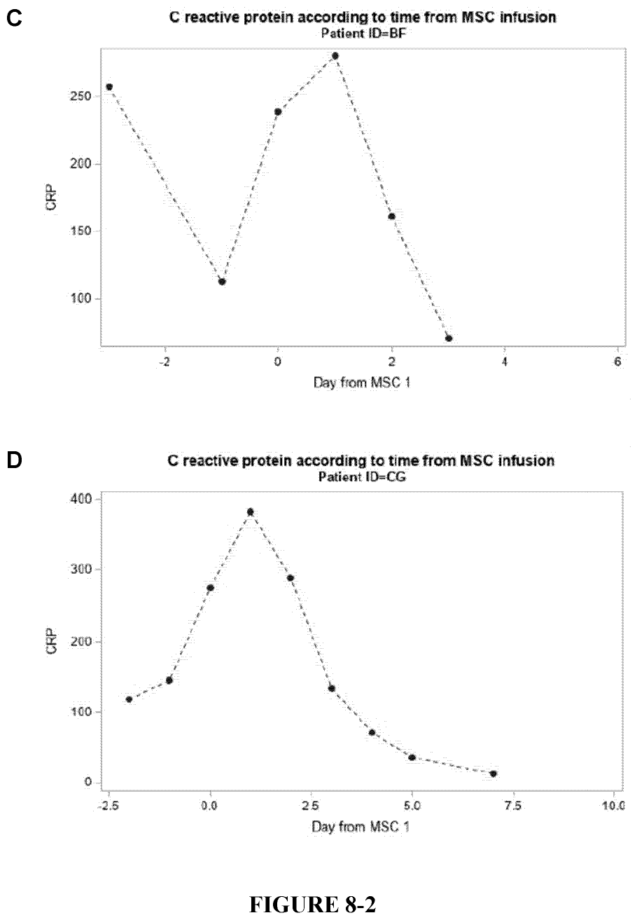

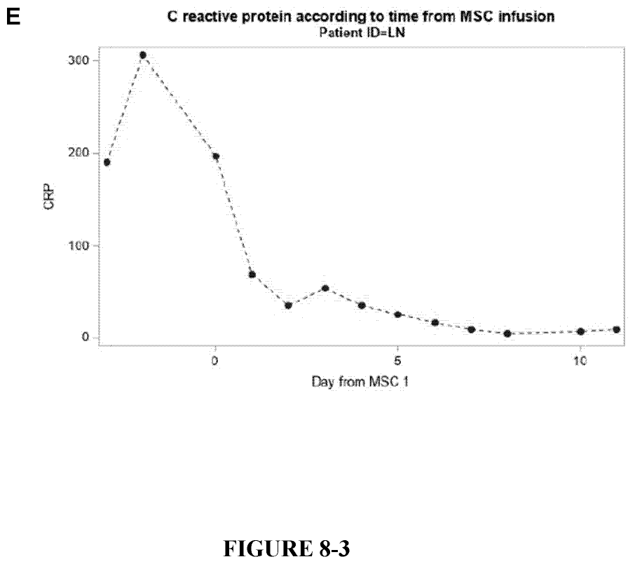

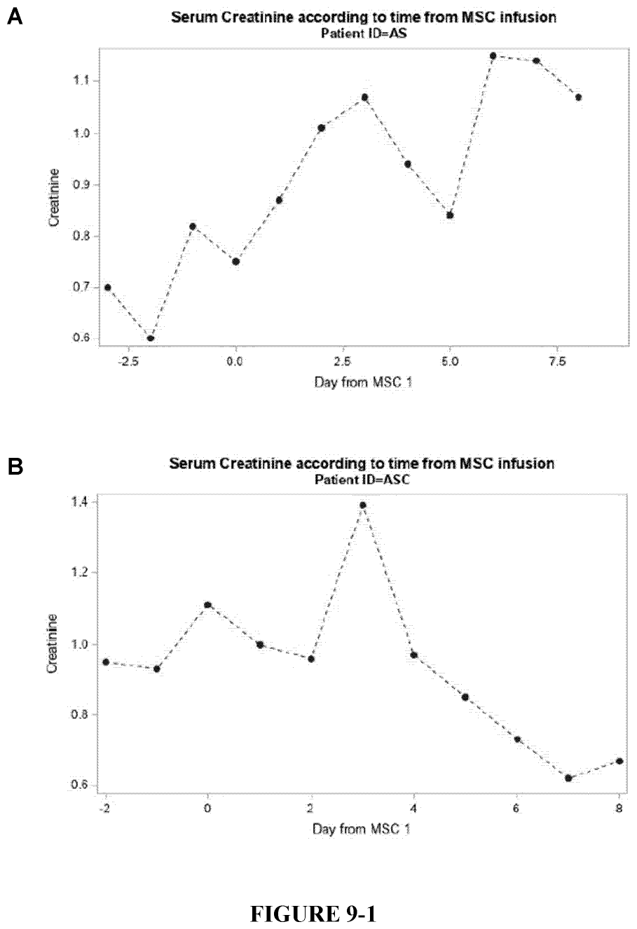

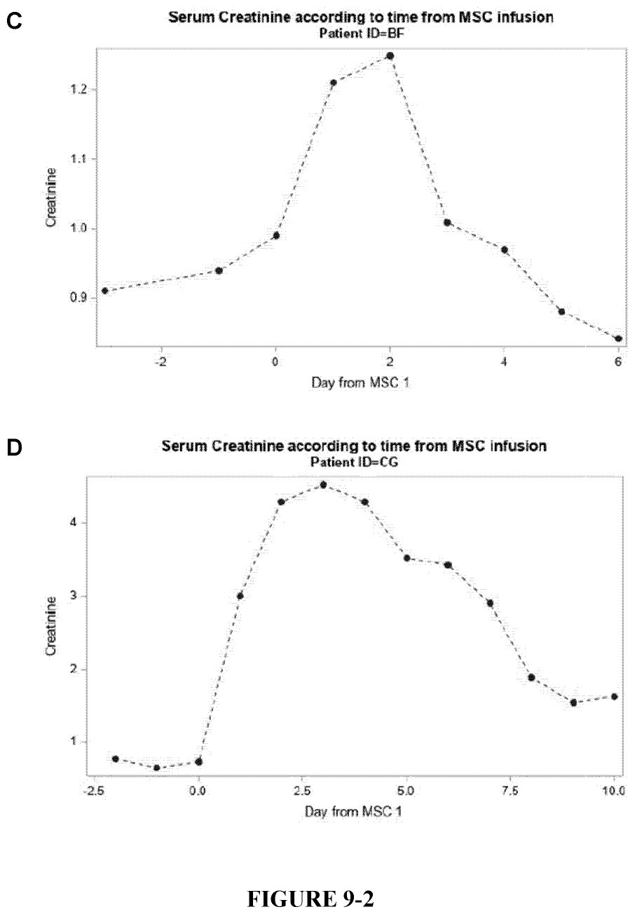

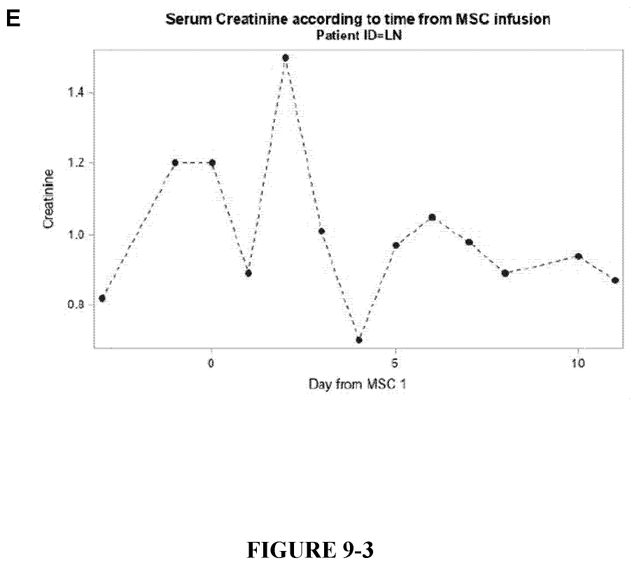

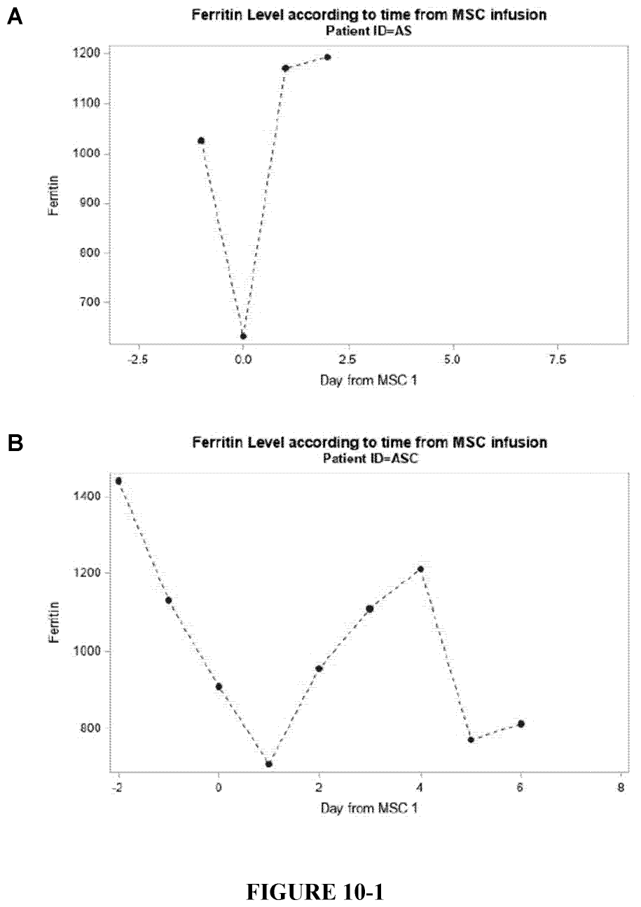

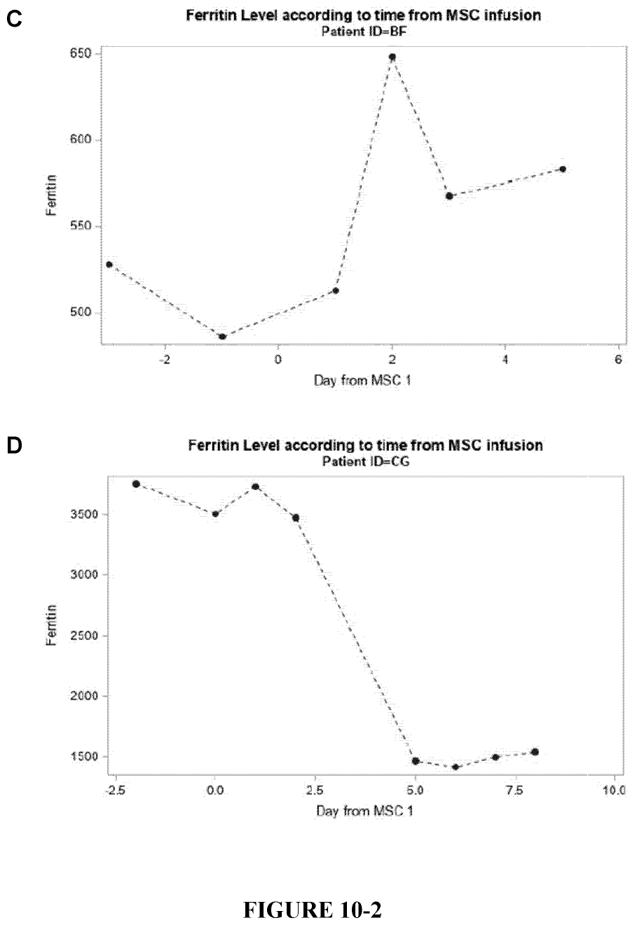

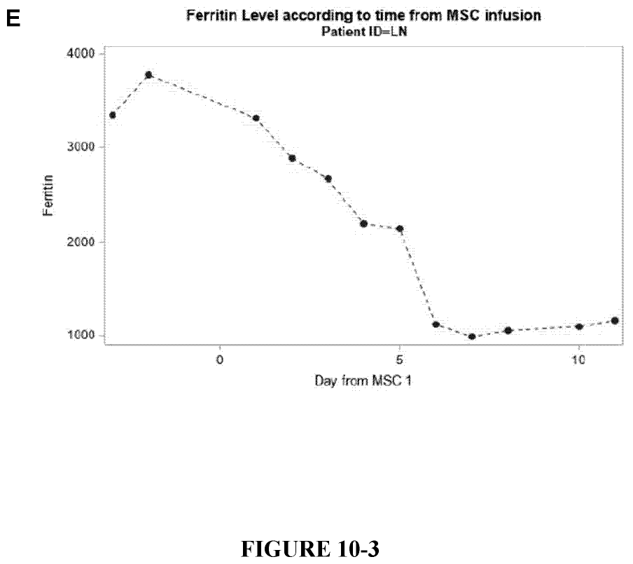

P/F ratios, CRP, creatinine and Ferritin levels are shown in 5 patients for 11 days post MSC infusion(s) (FIGS. 6 to 10). Day 0 is immediately before the first dose. All patients had second dose at day 2-5. P/F ratios generally increased over time and in some instances indicated improvement in ARDS grade. Circulating CRP levels generally decreased over time indicating reduced inflammation.

### Expanded Population

The patient population was expanded to 11 and subjects (Table 2) received two doses (intravenous) at 2 million cells/kg/infusion, 48-120 hours apart. Cell viability ranged between 78-90%.

TABLE 2

| | | | | Body | | |
|---|---|---|---|---|---|---|
| | | | | Mass | | P:F Nadir |
| | | Age, | Coexisting | Index, | APACHE | on Admission, |
| Patient | Gender | years | conditions | kg/m² | II Score* | mmHg** |
| 1 | Male | 60 | Obesity | 32.0 | 14 | 78 |
| 2 | Male | 39 | Obesity | 38.9 | 15 | 132 |
| 3 | Male | 35 | Obesity | 37.7 | 16 | 91 |
| 4 | Male | 60 | Obesity | 32.9 | 12 | 80 |
| | | | Asthma | | | |
| 5 | Male | 51 | Obesity | 34.4 | 13 | 133 |
| 6 | Female | 67 | Obesity | 37.8 | 14 | 106 |
| | | | Diabetes | | | |
| | | | Mellitus | | | |
| | | | Hypertension | | | |
| 7 | Male | 48 | Diabetes | 23.0 | 11 | 127 |
| | | | Mellitus | | | |
| 8 | Male | 58 | Hypertension | 25.2 | 14 | 144 |
| | | | Asthma | | | |
| 9 | Female | 34 | Obesity | 36.4 | 8 | 134 |
| 10 | Male | 50 | Obesity | 35.6 | 16 | 151 |
| | | | Diabetes | | | |
| | | | Mellitus | | | |
| | | | Hypertension | | | |
| 11 | Male | 60 | None | 23.6 | 17 | 71 |

### Clinical Outcomes

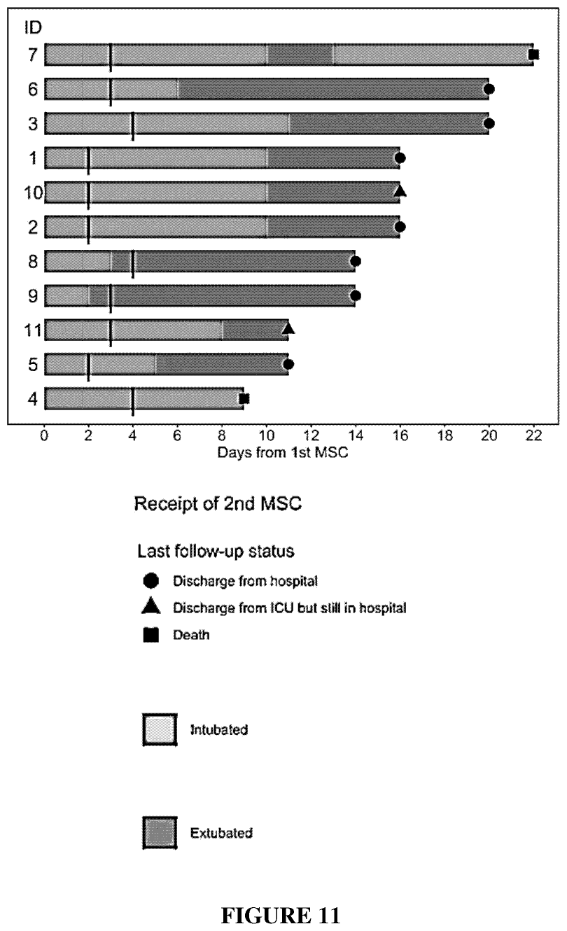

Clinical outcomes are shown in FIG. 11. All patients were followed until death or ICU discharge, and the ICU mortality was 18% (95% CI; 2-52%). Over the period of observation, ten (91%, 95% CI; 59-100%) patients were extubated. One patient (patient 7) was re-intubated three days after his extubation because of a sudden decompensation from presumed massive pulmonary embolism. Thrombolytic therapy resulted in improvement in oxygenation and hemodynamics but the patient ultimately died at day 23 post-infusion from septic shock while still requiring mechanical ventilation. At the end of the study period, nine (82%, 95% CI; 48-97%) patients were liberated from mechanical ventilation, nine (82%, 95% CI; 48-97%) were discharged from the intensive care unit and seven (64%, 95% CI; 31-89) were discharged from the hospital. Two remain hospitalized in stable condition on the medical floor. The median time to extubation from first infusion was ten days (IQR: 3-10 days). The median duration of mechanical ventilation was 12 days (IQR: 7-12 days).

### Physiologic Data

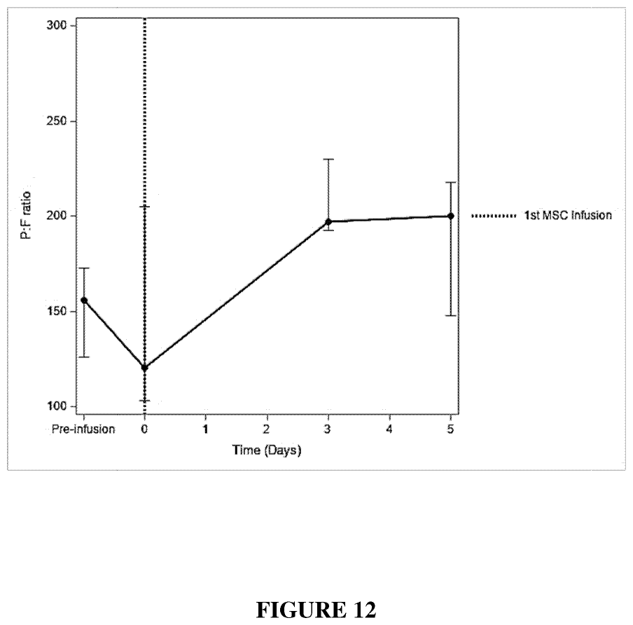

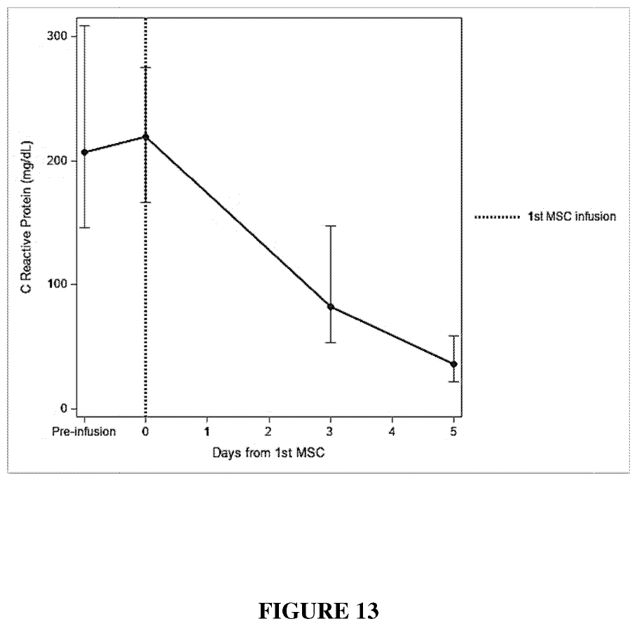

There was an improvement in oxygenation as assessed by the daily median P:F ratio (median difference+78 mmHg, p=0.002) from day 0 to day 3 (Table 3 and 4; FIG. 12). CRP levels decreased from day 0 to day 5 (median value 219.2 to 36.5 mg/L, p=0.002, FIG. 13, Tables 3 and 4). There was no change in fever curve or white blood cell count. Ferritin levels and SOFA scores did not significantly change from day 0 to day 3 or 5.

TABLE 3
*Values obtained within 24-48 hours of intubation.

| | Pre-infusion | | | | |
|---|---|---|---|---|---|
| Parameter | value* | Day 0 | Day 3 | Day 5 | p value** |
| Daily Maximal | 38.4 | 38.1 | 37.3 | 37.7 | 0.229 |
| Temperature, | (37.6-39.0) | (36.2-39.5) | (37.0-38.5) | (37.1-38.3) | |
| degrees Celsius | | | | | |
| White Blood Cell | 15.8 | 12.7 | 15.5 | 15.8 | 0.234 |
| Count, ×10⁹/L | (12-.0-19.6) | (8.6-19.0) | (11.0-17.1) | (10.8-20.1) | |
| C reactive protein, | 207.0 | 219.2 | 82.3 | 36.5 | <0.001 |
| mg/L | (145.5-308.6) | (166.4-275.1) | (53.2-147.6) | (22.1-58.5) | |
| Ferritin, mg/dL | 1027 | 909 | 719 | 704 | 0.061 |
| | (716-1755) | (632-1524) | (528-1411) | (554-1089) | |
| P:F ratio*** | 156 | 120 | 197 | 200 | <0.001 |
| | (125-172) | (103-205) | (192-230) | (148-218) | |
| SOFA score^{α} | 7.0 | 6.0 | 4.0 | 3.5 | 0.009 |
| | (6.0-10.0) | (3.0-7.0) | (3.0-7.0) | (2.5-6.0) | |

*Values obtained within 24-48 hours of intubation.
**Friedman's test
***P:F Ratio of the partial pressure of oxygen in arterial blood to the fraction of inspired oxygen
^{α}Sequential Organ Failure Assessment Score

TABLE 4

| | | Median Difference | | |
|---|---|---|---|---|
| Parameter | Comparison | (IQR) | | p value* |
| C reactive | Difference from | −141.8 | (−211.0, 183.6) | 0.032 |
| protein | day 0 to day 3 | | | |
| | Difference from | −176.2 | (−240.0, −41.2) | 0.002** |
| | day 0 to day 5 | | | |
| P:F ratio*** | Difference from | 78 | (3-154) | 0.002** |
| | day 0 to day 3 | | | |
| | Difference from | 49 | (30-166) | 0.004** |
| | day 0 to day 5 | | | |
| SOFA score^{α} | Difference from | −1 | (−8, 6) | 0.523 |
| | day 0 to day 3 | | | |
| | Difference from | −1 | (−8, 4) | 0.438 |
| | day 0 to day 5 | | | |

*Wilcoxon signed rank test
**Significant at an alpha < 0.008, using a Bonferroni correction for multiple comparisons
***P:F Ratio of the partial pressure of oxygen in arterial blood to the fraction of inspired oxygen
^{α}Sequential Organ Failure Assessment Score

### Safety

There were no infusion related adverse events.

### Ex-Vivo Culture-Expanded Adult Allogeneic Bone Marrow Derived Mesenchymal Stem Cells (MSCs), for the Treatment of Multi-System Inflammatory Syndrome (MIS)

A previously healthy boy presented with high fevers and was positive for COVID-19 antibodies but not the virus. He was initially treated for MIS with high-dose steroids, intravenous immunoglobulins, vasopressors, high-dose aspirin and anti-platelet medications. Despite these multiple therapeutics, he experienced deteriorating heart pump function as well as persistently elevated biomarkers for inflammation, cardiac congestion, and intravascular clotting of blood. Four days after hospital admission, the child was treated with a dose of intravenous mesenchymal stem cells (2×10⁶ cells/kg) followed by a second intravenous dose two days later.

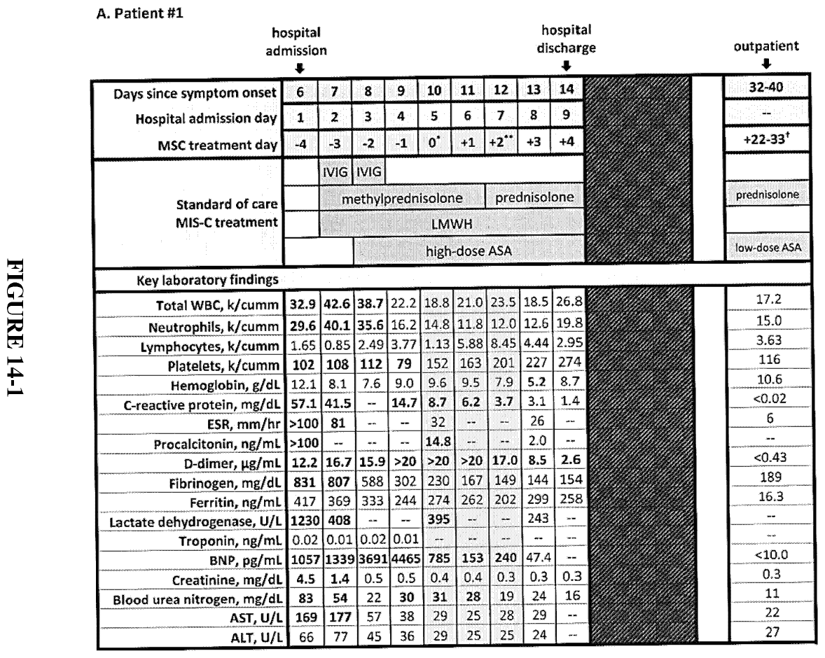

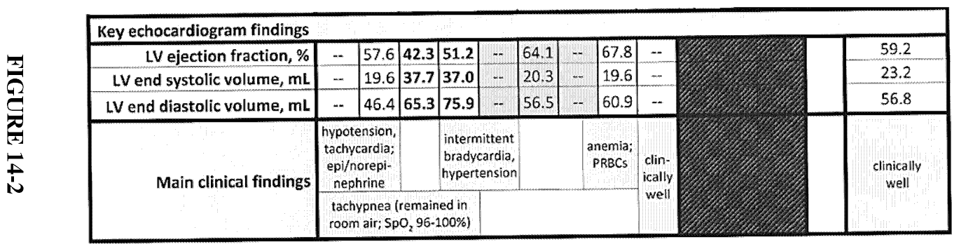

Over a period of three days in which the two MSC doses were administered, CRP, D-dimer and BNP levels decreased while LVEF % increased (Table 5; FIG. 14). Notable reductions in inflammatory biomarkers were also observed (FIG. 14). As shown in Table 5, D-dimer levels, a marker of major thrombosis and clotting, was dramatically reduced (from >20 to 2.6 μg/mL) and heart function was significantly improved with ejection fraction increasing from 51% to 68%. The child was subsequently discharged home.

TABLE 5
MSCs dosing @ 2 × 10⁶ cells/kg
4 year old male; previously healthy; 18 kg

| | | Dose #1 | | Dose #2 | |
|---|---|---|---|---|---|
| Day | −1 | 0 | 1 | 2 | 3 |
| hsCRP | 8.7 | 6.2 | 3.7 | 3 | 1.4 |
| D-Dimer ug/ml | >20 | 17 | — | — | 2.57 |
| LVEF % | 51 | 64 | — | 68 | — |
| BNP pg/ml | 785 | 153 | 240 | 49 | — |

A previously healthy girl was positive for COVID-19 antibodies but not the virus. She was initially treated for MIS with IV steroids, vasopressors, low-dose aspirin and intubated as a precaution. Two days after hospital admission, the child was treated with a dose of intravenous mesenchymal stem cells (2×10⁶ cells/kg) followed by a second intravenous dose two days later.

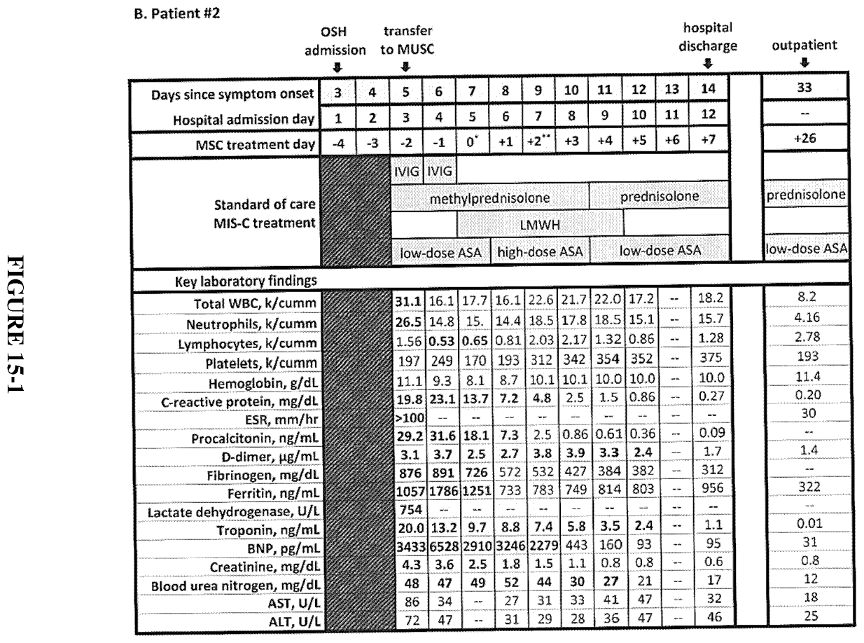

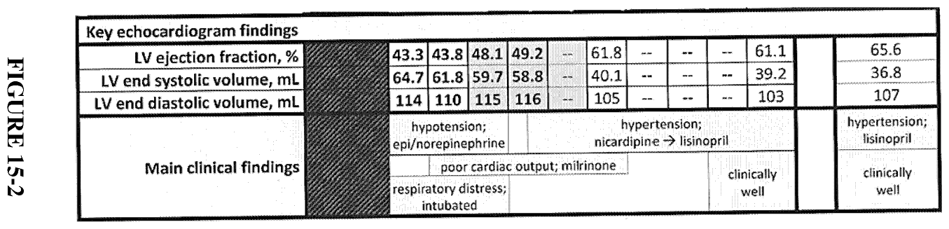

Over a period of four days in which the two MSC doses were administered, CRP, Troponin I, Ferritin, Creatinine, BUN and BNP levels decreased while LVEF % increased (Table 6; FIG. 15). Notable reductions in inflammatory biomarkers were also observed (FIG. 15). Reduced fibrinogen levels were observed after the second dose of cells. Furthermore, as shown in Table 6, heart function was significantly improved with ejection fraction increasing from 43.3% to 61.8%.

TABLE 6
MSCs dosing @ 2 × 10⁶ cells/kg
10 year old female; previously healthy; 58 kg

| | | Dose #1 | | Dose #2 | | | |
|---|---|---|---|---|---|---|---|
| Day | −2 | 0 | 1 | 2 | 3 | 4 | 5 |
| hsCRP | 23.0 | — | 7.2 | 4.5 | 2.5 | 1.49 | 0.86 |
| Troponin I | 13 | — | — | — | 7.4 | — | — |
| LVEF % | 43.3 | 48.1 | 49.2 | — | 61.8 | — | — |
| BNP | 6500 | 2900 | — | 2279 | 443 | 160 | 93 |
| Ferritin | 1700 | 1250 | 732 | 782 | 748 | 814 | 803 |
| Creatinine | 4.3 | 1.5 | — | 1.1 | 0.8 | 0.8 | 0.6 |
| BUN | 46 | — | — | 44 | 30 | 27 | 21 |
| Fibrinogen | — | — | 572 | 532 | 427 | 384 | 382 |

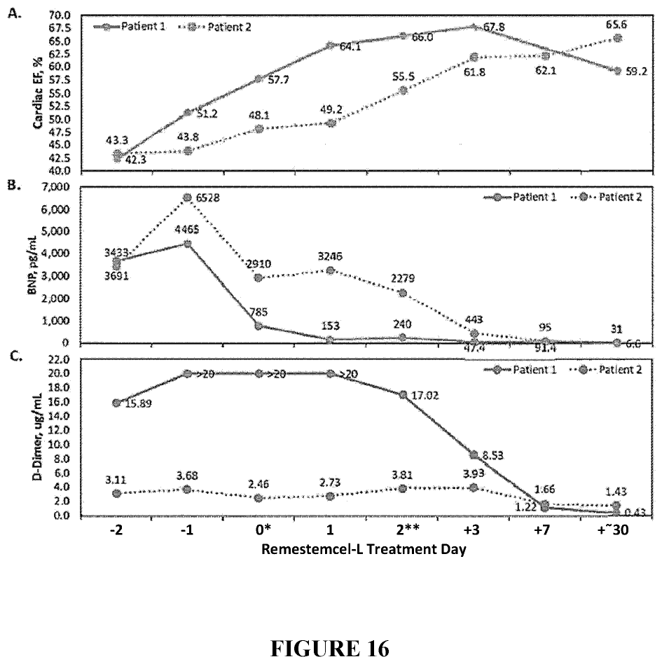

As noted in the above Tables, following administration of cell therapy, both children showed rapid normalization of their LV ejection fraction and B-type natriuretic protein as well as improvement in D-dimer that were temporally-associated with remestemcel-L treatment (FIG. 16). In addition, serial echocardiographic imaging showed reduction in the severity of pan-valvular regurgitation as well as increases in LV end systolic volume. The latter observation is consistent with improvement in LV contractile state that was temporally-associated with the cell therapy administration.

### Ex-Vivo Culture-Expanded Adult Allogeneic Bone Marrow Derived Mesenchymal Stem Cells (MSCs), for the Treatment of Hyperinflammation in Transplant Recipient

An male adult had a colon resection and transplant for severe Crohn's disease. The patient subsequently developed bacterial ARDS and symptoms indicative of transplant rejection. The patient was also suffering from COPD with CT findings of centrilobular emphysema in their upper lobes. The patient was not responsive to any treatment including treatment with Oral Vancomycin (started due to history of recurrent Clostridium difficile infection) and oral Augmentin (started for pneumonia). Colonoscopy revealed small bowel mucosa with patchy active inflammation, architectural distortion, and focally increased apoptotic activity (5/10 consecutive crypts), indeterminant for acute cellular rejection. Chest CT showed new findings of multifocal peribronchial nodular and dependent consolidative opacities, predominantly in the lower lobes, suspicious for pneumonia. A further colonoscopy was performed showing Ileum was normal, and colonic mucosa was erythematous throughout. Pathology again returned indeterminant for acute cellular rejection, but inflammatory changes were present.

The patient was subsequently administered 2 infusions of intravenous mesenchymal stem cells (2×10⁶ cells) followed by direct administration (endoscopic) of 150 million MSCs to the colon wall. The patients respiratory ARDS cleared post cell therapy and the patient was discharged home, off oxygen, and having 3-4 bowel movements a day. Semi-solid in consistency. Follow-up a fortnight later revealed, drastic improvement in clinical signs, minimal inflammation of the Ileum, no background inflammation in the colon and no evidence of transplant rejection. No shortness of breath was reported, diarrhea was back to baseline (10-12× occurrence), no fevers or SOB was reported and no supplemental O₂ use was necessary. Bronchial lavage cultures were also negative for infection. Further follow-up revealed that the patent was at home and feeling well.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All publications discussed above are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

The present application claims priority from AU2020901052 filed 3 April 2020, AU2020901124 filed 8 Apr. 2020, AU2020902312 filed 6 Jul. 2020, AU2020902425 filed 14 Jul. 2020, AU2020903041 filed 25 Aug. 2020, AU2020903694 filed 12 Oct. 2020 and, AU2020904312 filed 23 Nov. 2020, the disclosures of which are incorporated herein by reference.

1. A method of treating or preventing hyperinflammation in a human subject in need thereof, the method comprising administering to the subject a composition comprising mesenchymal lineage precursor or stem cells (MLPSCs).

2. The method of claim 1, wherein the hyperinflammation is caused by a viral infection.

3. The method of claim 2 wherein the viral infection is caused by a rhinovirus, an influenza virus, a respiratory syncytial virus (RSV) or a coronavirus.

4. The method of claim 3 wherein the viral infection is caused by a coronavirus.

5. The method of claim 4 wherein the coronavirus is Severe Acute Respiratory Syndrome coronavirus (SARS-CoV), Middle East Respiratory Syndrome coronavirus (MERS-CoV) or COVID-19.

6. The method according to any one of claims 1 to 5, wherein the subject also has Acute Respiratory Distress Syndrome (ARDS).

7. The method of any one of claims 1 to 5, wherein the MLPSCs have been cryopreserved and thawed.

8. The method of any one of claims 1 to 7, wherein the MLPSCs are culture expanded from an intermediate cryopreserved MLPSCs population.

9. The method according to any one of claims 1 to 8, wherein the MLPSCs are culture expanded for at least about 5 passages.

10. The method of any one of claims 1 to 9, wherein the MLPSCs express at least 13 pg TNFR1 per million MLPSCs.

11. The method of any one of claims 1 to 10, wherein the MLPSCs express about 13 pg to about 44 pg TNFR1 per million MLPSCs.

12. The method of any one of claims 9 to 11, wherein said culture expansion comprises at least 20 or 30 population doublings.

13. The method of any one of claims 1 to 12, wherein the subject has MIS.

14. The method according to any one of claims 1 to 13, wherein the subject has viral myocarditis.

15. The method according to any one of claims 1 to 14, wherein the MLPSCs are mesenchymal stem cells (MSCs).

16. The method according to any one of claims 1 to 15, wherein the MLPSCs are allogeneic.

17. The method according to any one of claims 1 to 16, wherein the MLPSCs are modified to carry or express an anti-viral drug or thrombolytic agent.

18. The method according to any one of claims 1 to 17 which comprises administering between 1×10⁷ and 2×10⁸ cells per dose.

19. The method according to any one of claims 1 to 18 which comprises administering about 1×10⁸ cells per dose.

20. The method according to claim 19, wherein the subject receives two doses.

21. The method according to any one of claims 1 to 20, wherein the subjects circulating CRP levels decrease after treatment.

22. The method according to any one of claims 1 to 21, wherein subjects white blood cell count decreases after treatment.

23. The method according to any one of claims 1 to 22, wherein the subjects FiO2 levels decrease after treatment and/or the subjects P/F ratio increases after treatment.

24. The method according to any one of claims 1 to 24, wherein the subjects D-dimer level is reduced after treatment.

25. The method of claim 24, wherein the subjects D-dimer level is reduced to below 5 μg/ml.

26. The method according to any one of claims 1 to 25, wherein the subjects BNP level is reduced after treatment.

27. The method according to any one of claims 1 to 26, wherein the subjects LVEF % is increased after treatment.

28. The method according to any one of claims 1 to 27, wherein the composition further comprises Plasma-Lyte A, dimethyl sulfoxide (DMSO), human serum albumin (HSA).

29. The method according to any one of claims 1 to 28, wherein the composition further comprises Plasma-Lyte A (70%), DMSO (10%), HSA (25%) solution, the HSA solution comprising 5% HSA and 15% buffer.

30. The method according to any one of claims 1 to 29, wherein the composition comprises greater than 6.68×10⁶ viable cells/mL.

31. A method of treating or preventing multi-system inflammatory syndrome (MIS) in a human subject in need thereof, the method comprising administering to the subject a composition comprising mesenchymal lineage precursor or stem cells (MLPSCs).

32. The method of claim 31, wherein the subject is a child.

33. A method of treating or preventing a disease associated with elevated D-dimer levels in a human subject in need thereof, the method comprising administering to the subject a composition comprising mesenchymal lineage precursor or stem cells (MLPSCs).

34. The method of claim 33, wherein the disease is thrombosis or pulmonary embolism.

35. The method of claim 33 or 34, wherein treatment reduces the subjects D-dimer level below 15 μg/ml.

36. The method of claim 33 or 34, wherein treatment reduces the subjects D-dimer level below 5 μg/ml.

37. The method of claim 34, wherein the thrombosis is arterial thrombosis.
